(19) United States
(12) Patent Application Publication
(10) Pub. No.: US 2023/0372434 A1
(43) Pub. Date: Nov. 23, 2023

(54) METHOD OF TREATING FIBROSIS WITH A COMBINATION THERAPY

(71) Applicant: COHBAR, INC., Menlo Park, CA (US)

(72) Inventor: Kenneth Cundy, Atherton, CA (US)

(21) Appl. No.: 18/031,018

(22) PCT Filed: Oct. 21, 2021
(86) PCT No.: PCT/US21/55983

§ 371 Date: Apr. 10, 2023

Related U.S. Application Data
(60) Provisional application No. 63/094,592, filed on Oct. 21, 2020.

(51) Int. Cl. A61K 38/10; A61K 31/496; A61P 11/00; A61K 31/4418

(52) U.S. Cl. CPC A61K 38/10; A61K 31/496; A61P 11/00; A61K 31/4418

(57) ABSTRACT

The disclosures herein relate to materials and methods and combinations effective for the treatment of fibrosis.

### TECHNICAL FIELD

The present invention is directed to the treatment of fibrosis, for example, idiopathic pulmonary fibrosis (IPF). More particularly, the disclosure relates to a combination of agents effective in the treatment of fibrosis.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ACII (Text) file named “54076_Seqlisting.txt” created on Oct. 15, 2021, and 20,381 bytes in size. To the extent differences exist between information/description of sequences in the specification and information in the Sequence Listing, the specification is controlling.

### BACKGROUND

Idiopathic pulmonary fibrosis (“IPF”) is a chronic, progressive, and lethal fibrotic disease of unclear etiology characterized by scaring (fibrosis) of the lung parenchyma and loss of lung function, including breathing. Multiple: molecular pathways are aberrantly expressed in idiopathidiopathic pulmonary fibrosis tissue including directly pro-fibrotic mediators such as TGFb, IL13, and FGF. Symptoms typically include gradual onset of shortness of breath and a dry, chronic cough. Other symptoms include chest pain and fatigue. Patients with IPF generally have a five year survival rate after the primary diagnosis of around 50%, The causes of IPF are not completely understood.

There is currently no cure for IPF and no procedures or medications that can remove the scarring from the lungs. Conventional treatment of IPF tends to focus on slowing the progression of the lung scarring. Such treatment includes pulmonary rehabilitation, supplemental oxygen, and/or use of medications like pirfenidone or nintedanib. Lung transplantation is also an option in severe cases. The bleomycin (BLM) murine model is probably the most accepted model of pulmonary fibrosis. Intratracheal administration of Neomycin effectively mimics the chronic aspect of pulmonary fibrosis, as well as other characteristics including the presence of Hyperplastic alveolar epithelial cells. (Mouratis et al., Current Opinion in Pulmonary Medicine: September 2011, Vol 17(5): 355-361). BLM-induced fibrosis in mice constitutes an animal model of IPF with high degree of similarity to the characteristics of lung fibrosis described in human idiopathic pulmonary fibrosis.

Steroids and immunosuppressant agents are not effective for IPF. Currently, anti-fibrosis agents, such as pirfenidone, are used in the clinical practice, however, the efficacy is limited and adverse effects in digestive organs and photo toxicity are observed. The present invention provides a novel use of a combination of anti-fibrosis therapies, for the treatment and/or alleviation of IPF.

### SUMMARY

Disclosed is a method for treating fibrosis comprising administering to the patient a pharmacologically effect amount of a combination of antifibrotic agents. The present disclosure moreover includes pharmaceutical compositions comprising combinations of antifibrotic agents.

Embodiments described herein as methods also can be described as “uses,” or products for specific uses and/or uses of products for manufacture of a medicament having a specific use, and all such uses are contemplated as embodiments. Likewise, compositions described herein as having a “use” can alternatively be described as processes or methods of using, which are contemplated as embodiments. By way of example, aspects described as methods of treatment have, as alternative embodiments, uses for treatment and uses for manufacture of a medicament for treatment.

Also described herein are peptides and peptide-related compositions with properties that make the peptides useful for treating fibrosis and other conditions.

The headings herein are for the convenience of the reader and not intended to be limiting. Other aspects of the invention will be apparent from the detailed description and claims that follow.

### DETAILED DESCRIPTION

In one aspect, methods of treating diseases relating to fibrosis are disclosed.

In one embodiment, a method for treating fibrosis is disclosed, comprising administering to a patient in need of such treatment, a pharmacologically effect amount of a combination of a first antifibrotic agent and a peptide antifibrotic agent selected from a peptide of amino acid sequences of Formula I:

(SEQ ID NO: 1)
X¹-R-X²-IR-X³-X⁴-L-X⁵-X⁶-G-X¹⁴-X⁷-G-X⁸-X⁹ (I)

wherein X¹ is absent or if present is an amino acid having a polar side chain or a non-polar side chain; X² is an amino acid having a non-polar side chain; X³ is an amino acid having a non-polar side chain; X⁴ is an amino acid having a polar side chain; X⁵ is an amino acid having a polar side chain or a non-polar side chain; X⁶ is an amino acid having a polar side chain or a non-polar side chain; X⁷ is an amino acid having a polar side chain or a non-polar side chain; X⁸ is an amino acid having a polar side chain; X⁹ is absent or is -X¹⁰-X¹¹-X¹²-X¹³; wherein X¹⁰ is an amino acid having a non-polar side chain; X¹¹ is an amino acid having a non-polar side chain; X¹² is absent or if present is an amino acid having a polar side chain or a non-polar side chain; and X¹³ is absent or if present is an amino acid having a polar side chain, provided X¹³ is absent if X¹² is absent; and X¹⁴ is an amino acid having a polar side chain or a non-polar side chain; or C-terminal acids or aminds and/or N-acetyl derivatives thereof; or pharmaceutically acceptable salts thereof.

In one embodiment, a method for treating fibrosis is disclosed, comprising administering to a patient in need of such treatment, a pharmacologically effect amount of a combination of a first antifibrotic agent and a second antifibrotic agent selected from a peptide of amino acid sequences of Formula I wherein X¹ is absent or is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dI), F, (dF), W, (dW), P, (dP), M and (dM); X² is selected from G, A, (dA), V, (dV), L, (dL), I, (dI), F, (dF), W, (dW), P, (dP), M and (dM); X³ is selected from G, A, (dA), V, (dV), L, (dL), I, (dI), F, (dF), W, (dW), P, (dP), Nle, M and (dM); X⁴ is selected from D, (dD), E, (dE), K, (dK), R, (dR), H, (dH), N, (dN), Q, (dQ), S, (dS), T, (dT), Y, (dY), C and (dC); X⁵ is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dI), F, (dF), W, (dW), P, (dP), M and (dM); X⁶ is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dI), F, (dF), W, (dW), P, (dP), M and (dM); X⁷ is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dI), F, (dF), W, (dW), P, (dP), M and (dM); X⁸ is selected from D, (dD), E, (dE), K, (dK), R, (dR), H, (dH), N, (dN), Q, (dQ), S, (dS), T, (dT), Y, (dY), C and (dC); and X⁹ is absent or is -X¹⁰-X¹¹-X¹²-X¹³, wherein X¹⁰ is selected from G, A, (dA), V, (dV), L, (dL), I, (dI), F, (dF), W, (dW), P, (dP), M and (dM); X¹¹ is selected from G, A, (dA), V, (dV), L, (dL), I, (dI), F, (dF), W, (dW), P, (dP), M and (dM); X¹² is absent or is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dI), F, (dF), W, (dW), P, (dP), M and (dM); and X¹³ is absent or is selected from D, (dD), E, (dE), K, (dK), R, (dR), H, (dH), N, (dN), Q, (dQ), S, (dS), T, (dT), Y, (dY), C and (dC); and and X¹⁴ is selected from D, (dD), E, (dE), K, (dK), R, (dR), H, (dH), N, (dN), Q, (dQ), S, (dS), T, (dT), Y, (dY), C, (dC), G, A, (dA), V, (dV), L, (dL), I, (dI), F, (dF), W, (dW), P, (dP), M and (dM); or pharmaceutically acceptable salts thereof.

In one embodiment, a method for treating fibrosis is disclosed, comprising administering to a patient in need of such treatment, a pharmacologically effect amount of a combination of a first antifibrotic agent and a second antifibrotic agent selected from a peptide of amino acid sequences of Formula I:

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK ,-LAGK; or -L(dA); and X¹⁴ is N or L; or C-terminal acids or amides, or N-acetyl derivatives thereof; or pharmaceutically acceptable salts thereof.

In one embodiment, a method for treating fibrosis is disclosed, comprising administering to a patient in need of such treatment, a pharmacologically effect amount of a combination of a first antifibrotic agent and a second antifibrotic agent selected from a peptide of amino acid sequences of any one of SEQ ID NO: 1-38.

In one embodiment, a combination is disclosed of a first anti-fibrotic agent and a second antifibrotic agent selected from a peptide of amino acid sequences of Formula I

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK ,-LAGK; or -L(dA); and X¹⁴ is N or L; or C-terminal acids or amides, or N-acetyl derivatives thereof; or pharmaceutically acceptable salts thereof.

In one embodiment, antifibrotic agents are selected from anti-LOXI:2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-6, αᵥβ₁/αₐβ₆ inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxdant), MK-2 inlibitors, Heat Shock Protein 47 (HSP47) gene therapies, Jun N-terminal kinase (JNK) 1/2 inhibitors, JNK1 inhibitors, NOX 1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and. LoxL2 inhibitors.

In one embodiment, antifibrotic agents are selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-6, αᵥβ₁/αᵥβ₆ inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, IRE-1 inhibitors, P2X3 antagonists, SRC inhibitors, Jun N-terminal kinase (JNK) 1/2 inhibitors, JNK1 inhibitors, NOX1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.

In one embodiment, the antifibrotic agents is a PDGF receptor inhibitor or a TGF-β inhibitor. In one embodiment, the TGF-β inhibitor is selected from vactosertib (TEW7197), pirfenidone and galunisertib. In one embodiment, the PDGF receptor inhibitor is selected from nintedanib, sunitinib, imatinib and sorafenib.

In one embodiment, the antifibrotic agent is one or more agents selected from pirfenidone and nintedanib.

In one embodiment, antifibrotic agents are selected from nintedanib, pirfenidone, indolinone, Simtuzumab, (Gilead, GS-6624), IW001 (ImmuneWorks), PRM-151 (Promedior), tanzisertib (Celgene, CC-930), imatinib, STX-100 (Biogen), dectrekumab (Novartis, QAX576), pamrevlumab (FibroGen), carlumab (Janssen, CNTO-888), SM-04646 (Samumed), N-acetylcysteine (NAC), CC-90001 (Celgene), BMS-986,020 (Bristol Myers Squibb), BMS-986,278 (Bristol Myers Squibb), BBT-877 (Bridge Biotherapeutics and Boehringer Ingelheim), GLPG1690 (Galapagos), BI 1015550 (Boehringer Ingelheim), Gefapixant, PBI-4050 (ProMetic), tipelukast (MediciNova), PAT-1251, lanalumab (Novartis), BLD-2660 (Blade), PLN-74809 (Pliant), ND-L02-s0201 (Nitto Denko), Setanaxib (GKT137831) (Genkyotex), KD025 (Kadmon), and GB0139 (Galecto).

In one embodiment, antifibrotic agents are selected from nintedanib, pirfenidone, BBT-877 (Bridge Biotherapeutics and Boehringer Ingelheim), GLPG1690 (Galapagos), BMS-986,020 (Bristol Myers Squibb), BMS-986,278 (Bristol Myers Squibb), Pamrevlumab (FibroGen), N-acetyl cysteine (NAC), PRM-151 (Promedior), lanalumab (Novartis), BLD-2660 (Blade), PLN-74809 (Pliant), ND-L02-s0201 (Nitto Denko), CC-90001 (Celgene), tipelukast (MediciNova, MN-001), Setanaxib (Genkyotex, GKT137831), KD025 (Kadmon), and GB0139 (Galecto).

In one embodiment, antifibrotic agents are selected from nintedanib, pirfenidone, indolinone, simtuzumab, (Gilead, GS-6624), IW001 (ImmuneWorks), PRM-151 [recombinant human pentraxin-2 protein, Promedior), tanzisertib (Celgene), imatinib, STX-100 (Biogen), dectrekumab (Novartis, QAX576), pamrevlumab (FibroGen), carlumab (Janssen, CNTO-888), SM-04646 (Samumed), N-acetylcysteine (NAC), BMS-986,020 (Bristol Myers Squibb), BMS-986,278 (Bristol Myers Squibb), BBT-877 (Bridge Biotherapeutics and Boehringer Ingelheim), GLPG1690 (Galapagos), BI 1015550 (Boehringer Ingelheim), Gefapixant, Setogepram (ProMetic, PBI-4050), tipelukast (MediciNova), GB 2064 (GALECTO, PAT-1251), lanalumab (Novartis), BLD-2660 (Blade), PLN-74809 (Pliant), ND-L02-s0201 (Nitto Denko), setanaxib (GKT137831) (Genkyotex), belumosudil (KD025, Kadmon), GB0139 (Galecto), BNC 1021 (BONAC/Toray, TRK-250), ORIN1001 (Fosun), sildenafil, macitentan, bosentan, lebrikizumab, valganciclovir, letemovir, minocycline, gefapixant, zileuton, NIP292 (CR Pharma), voxelotor (GBT446), HEC825, HEC6840, saracatinab, and CC90001 (BMS).

In one embodiment, antifibrotic agents are selected from ib, pirfenidone, indolinone, simtuzumab, tanzisertib, imatinib, dectrekumab, pamrevlumab, carlumab, N-acetylcysteine (NAC), gefapixant, Setogepram, tipelukast, lanalumab, setanaxib, belumosudil, sildenafil, macitentan, bosentan, lebrikizumab, valganciclovir, letemovir, minocycline, gefapixant, zileuton, voxelotor and saracatinab.

In one embodiment, antifibrotic agents are selected from

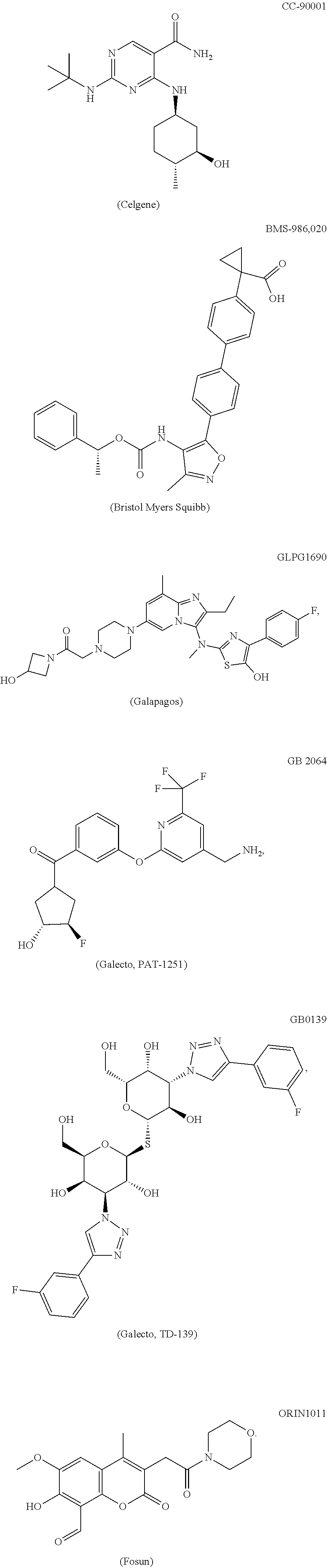 [SMILES: C[C@@H]1CC[C@@H](Nc2nc(NC(C)(C)C)ncc2C(N)=O)C[C@H]1O ; Cc1noc(-c2ccc(-c3ccc(C4(C(=O)O)CC4)cc3)cc2)c1NC(=O)O[C@H](C)c1ccccc1 ; CCc1nc2c(C)cc(N3CCN(CC(=O)N4CC(O)C4)CC3)cn2c1N(C)c1nc(-c2ccc(F)cc2)c(O)s1 ; NCc1cc(Oc2cccc(C(=O)C3C[C@@H](O)[C@H](F)C3)c2)nc(C(F)(F)F)c1 ; OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O ; COc1cc2c(C)c(CC(=O)N3CCOCC3)c(=O)oc2c(C=O)c1O]

In one embodiment, a peptide of any one or more of the amino acid sequences set forth in any one of SEQ ID NO: 32-38 are disclosed.

An embodiment comprises a peptide of the amino acid sequence MRVIRMCLGVGLLGDLAG (SEQ ID NO: 2). In some embodiments a peptide is in a modified form of SEQ ID NO: 2 comprising up to 10 amino acid modifications relative to SEQ ID NO: 2. In some embodiments a peptide is in a modified form of SEQ ID NO: 2 comprising up to 8 amino acid modifications relative to SEQ ID NO: 2, the modification(s) being in one or more of the positions 1, 2, 3, 4, 5, 6, 7, 8, 9, 10, 11, 12, 13, 14, 15, 16, 17 or 18, wherein the amino acid numbering corresponds to SEQ ID NO: 2. In some embodiments a peptide is in a modified form of SEQ ID NO: 2 comprising up to 6 amino acid modifications relative to SEQ ID NO: 2, the modification(s) being in one or more of the positions 1, 9, 13, 15, 17 or 18, wherein the amino acid numbering corresponds to SEQ ID NO: 2.

An embodiment comprises a peptide selected from MRVIRMCLGVGLLGDLAG (SEQ ID NO: 2); RVIRMCLGVGLLGDLAG (SEQ ID NO: 3); RVIRMCLGVGLLGDL(dA)G (SEQ ID NO: 4); RVIRMCLNVGLLGEL(dA)G (SEQ ID NO: 5); RVIR(Nle)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNLGEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIRMCLGVGLLGDLAG (SEQ ID NO: 16); RVIRMCLGVGLLGDLAGK{PEG12} (SEQ ID NO: 17); {PEG12}KRVIRMCLNVGLLGEL(dA)E (SEQ ID NO: 18); RVIRMCLNVGLEGEL(dA) (SEQ ID NO: 19); RVIRMCLNVGLNGEL(dA)E (SEQ ID NO: 20); RVIRMCLNVGLNGE (SEQ ID NO: 21); RVIRMCLNNGLNGEL(dA)G (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK{PEG12} (SEQ ID NO: 25); RVIRACLGVGLLGDL(dA)GK PEG12 (SEQ ID NO: 29); RVIR(Nle)CLGVGLLGDL(dA)GK (SEQ ID NO: 33); RVIR(Nle)CLGVGLLGDL(dA)GK{PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLLGDLAGK{PEG12} (SEQ ID NO: 38); or pharmaceutically acceptable salts thereof.

An embodiment comprising RVIRMCLGVGLLGDL(dA)G (SEQ ID NO: 4); RVIRMCLNVGLLGEL(dA)G (SEQ ID NO: 5); RVIR(Nle)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNLGEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIRMCLGVGLLGDLAG (SEQ ID NO: 16); RVIRMCLGVGLLGDLAGK{PEG12} (SEQ ID NO: 17); {PEG12}KRVIRMCLNVGLLGEL(dA)E (SEQ ID NO: 18); RVIRMCLNVGLEGEL(dA) (SEQ ID NO: 19); RVIRMCLNVGLNGEL(dA)E (SEQ ID NO: 20); RVIRMCLNVGLNGE (SEQ ID NO: 21); RVIRMCLNNGLNGEL(dA)G (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK{PEG12} (SEQ ID NO: 25); RVIRACLGVGLLGDL(dA)GK{PEG12} (SEQ ID NO: 29); RVIR(Nle)CLGVGLLGDL(dA)GK (SEQ ID NO: 33); RVIR(Nle)CLGVGLLGDL(dA)GK{PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLLGDLAGK{PEG12} (SEQ ID NO: 38); or pharmaceutically acceptable salts thereof.

In some embodiments the antifibrotic agents are combined and/or used in synergistically effective amounts.

In some embodioments, the pepide antifibrotic agents are prepared as described in international patent publication no. WO 2020/160003, incorporated herein by reference.

In some embodiments a peptide is represented by the peptides listed in Table 1.

TABLE 1

| Sequence | SEQ ID NO: |
|---|---|
| MRVIRMCLGVGLLGDLAG | 2 |
| RVIRMCLGVGLLGDLAG | 3 |
| RVIRMCLGVGLLGDL(dA)G | 4 |
| RVIRMCLNVGLLGEL(dA)G | 5 |
| RVIR(Nle)CLNVGLLGEL(dA)G | 6 |
| RVIRMSLNVGLLGEL(dA)G | 7 |
| RVIR(Nle)SLNVGLLGEL(dA)G | 8 |
| RVIRMCLNNGLLGEL(dA)G | 9 |
| RVIRMCLNVGNLGEL(dA)G | 10 |
| RVIRMCLNVGLNGEL(dA)G | 11 |
| RVIRMCLNVGLLGEL(dA)E | 12 |
| RVIRMSLNVGLEGEL(dA) | 13 |
| RVIR(Nle)SLNVGLEGEL(dA) | 14 |
| R(dA)IR(Nle)SLNVGLLGEL(dA) | 15 |
| {PEG12}KRVIRMCLGVGLLGDLAG | 16 |
| RVIRMCLGVGLLGDLAGK{PEG12} | 17 |
| {PEG12}KRVIRMCLNVGLLGEL(dA)E | 18 |
| RVIRMCLNVGLEGEL(dA) | 19 |
| RVIRMCLNVGLNGEL(dA)E | 20 |
| RVIRMCLNVGLNGE | 21 |
| RVIRMCLNNGLNGEL(dA)G | 22 |
| RVIRMCLNNGLNGEL(dA)E | 23 |
| {5-FAM}-RVIRMCLGVGLLGDLAG | 24 |
| {5-FAM}-RVIRMCLGVGLLGDLAGK{PEG12} | 25 |
| RVIRMCLGVGLLGDLAG | 26 |
| \| | |
| RVIRMCLGVGLLGDLAG | |
| RVIRMCLNVGLLGEL(dA)G | 27 |
| \| | |
| RVIRMCLNVGLLGEL(dA)G | |
| RVIRMCLGVGLLGDLAGK{PEG12} | 28 |
| \| | |
| RVIRMCLGVGLLGDLAGK{PEG12} | |
| RVIRACLGVGLLGDL(dA)GK{PEG12} | 29 |
| RVIRACLGVGLLGDL(dA)GK{PEG12} | 30 |
| \| | |
| RVIRACLGVGLLGDL(dA)GK{PEG12} | |
| RVIR(Nle)CLGVGLLGDL(dA)GK{PEG12} | 32 |
| \| | |
| RVIR(Nle)CLGVGLLGDL(dA)GK{PEG12} | |
| RVIR(Nle)CLGVGLLGDL(dA)GK | 33 |
| RVIR(Nle)CLGVGLLGDL(dA)GK{PEG12} | 34 |
| RVIRACLGVGLLGDL(dA)GK | 35 |
| RVIRACLGVGLLGDLAGK{PEG12} | 36 |
| \| | |
| RVIRACLGVGLLGDLAGK{PEG12} | |
| RVIRACLGVGLLGDLAGK | 37 |
| RVIRACLGVGLLGDLAGK{PEG12} | 38 |

In some embodiments, peptides disclosed herein comprise a sequence having at least 66% sequence identity to any one of amino acid sequences SEQ ID NO: 1-38. In certain embodiments, the % identity is selected from, e.g., at least 70%, at least 75%, at least 80%, at least 85%, at least 90%, or at least 95%, or more sequence identity to a given sequence. In certain embodiments, the % identity is in the range of, e.g., about 65% to about 70%, about 70% to about 80%, about 80% to about 85%, about 85% to about 90%, or about 90% to about 95%; between about 70% and about 80%, between about 80% and about 90% and between about 90% and about 99% sequence identity.

In certain embodiments, the peptide does not comprise the sequence set forth in SEQ ID NO: 2 or SEQ ID NO: 3.

In exemplary embodiments, the peptide or peptide analog is a C-terminal acid or amide, or an N-acetyl derivative thereof.

In exemplary embodiments, the peptide or peptide derivative is a PEG, acetyl, biotin or fatty acid derivative thereof In exemplary embodiments, the peptide derivative includes PEG-12, acetyl, FAM or palmityl.

Peptides of the disclosure include peptides that have been modified in any way and for any reason, for example, to: (1) reduce susceptibility to proteolysis, (2) alter binding affinities, and (3) confer or modify other physicochemical or functional properties. For example, single or multiple amino acid substitutions (e.g., equivalent, conservative or non-conservative substitutions, deletions or additions) may be made in a sequence.

A conservative amino acid substitution refers to the substitution in a peptide of an amino acid with a functionally similar amino acid having similar properties, e.g., size, charge, hydrophobicity, hydrophilicity, and/or aromaticity. The following six groups each contain amino acids that are conservative substitutions for one another are found in Table 2.

TABLE 2

| | |
|---|---|
| i. | Alanine (A), Serine (S), and Threonine (T) |
| ii. | Aspartic acid (D) and Glutamic acid (E) |
| iii. | Asparagine (N) and Glutamine (Q) |
| iv. | Arginine (R) and Lysine (K) |
| v. | Isoleucine (I), Leucine (L), |
| | Methionine (M), and Valine (V) |
| vi. | Phenylalanine (F), Tyrosine (Y), |
| | and Tryptophan (W) |

Additionally, within the meaning of the term “equivalent amino acid substitution” as applied herein, one amino acid may be substituted for another, in one embodiment, within the groups of amino acids indicated herein below:
1. Amino acids with polar side chains (Asp, Glu, Lys, Arg, His, Asn, Gln, Ser, Thr, Tyr, and Cys,)
2. Amino acids with small nonpolar or slightly polar residues (Ala, Ser, Thr, Pro, Gly);
3. Amino acids with non-polar side chains (Gly, Ala, Val, Leu, Ile, Phe, Trp, Pro, and Met)
4. Amino acids with large, aliphatic, nonpolar residues (Met, Leu, Ile, Val, Cys, Norleucine (Nle), homocysteine)
5. Amino acids with aliphatic side chains (Gly, Ala Val, Leu, Ile)
6. Amino acids with cyclic side chains (Phe, Tyr, Trp, His, Pro)
7. Amino acids with aromatic side chains (Phe, Tyr, Trp)
8. Amino acids with acidic side chains (Asp, Glu)
9. Amino acids with basic side chains (Lys, Arg, His)
10. Amino acids with amide side chains (Asn, Gln)
11. Amino acids with hydroxy side chains (Ser, Thr)
12. Amino acids with sulphur-containing side chains (Cys, Met),
13. Neutral, weakly hydrophobic amino acids (Pro, Ala, Gly, Ser, Thr)
14. Hydrophilic, acidic amino acids (Gln, Asn, Glu, Asp), and
15. Hydrophobic amino acids (Leu, Ile, Val).

The present disclosure provides peptides comprising peptidomimetic compounds having further improved stability and cell permeability properties. Some embodiments comprise a peptide according to any of SEQ ID NO: 1-38, wherein one of more peptide bonds (—CO—NH—) within the peptide may be substituted, for example, by N-methylated amide bonds (—N(CH₃)—CO—), ester bonds (—C(=0)-0-), ketomethylene bonds (—CO—CH₂—), sulfinylmethylene bonds (—S(=0)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), or retro amide bonds (—NH—CO—), peptide derivatives (—N(R^{x})—CH₂—CO—), wherein R^{x} is the “normal” side chain, naturally present on the carbon atom. These modifications can occur at any of the bonds along the peptide chain and even at several (2-3) bonds at the same time.

The peptides of some embodiments are preferably utilized in a linear form, although it will be appreciated that in cases where cyclization does not severely interfere with peptide characteristics, cyclic forms of the peptide can also be utilized and are contemplated as embodiments.

Size variants of the peptides described herein are specifically contemplated. Exemplary peptides are composed of 6 to 50 amino acids. All integer subranges of 6-50 amino acids (e.g., 7-50 aa, 8-50 aa, 9-50 aa, 6-49 aa, 6-48 aa, 7-49 aa, and so on) are specifically contemplated as genera of the invention; and all interger values are contemplated as species of the invention. In exemplary embodiments, the peptide comprises at least seven or eight amino acids connected via peptide bonds. In exemplary aspects, the peptide is at least about 9 amino acids in length, about 10 amino acids in length, about 11 amino acids in length, about 12 amino acids in length, or about 13 amino acids in length. In exemplary aspects, the peptide is at least about 14 amino acids in length, about 15 amino acids in length, about 16 amino acids in length, or about 17 amino acids in length. In exemplary aspects, the peptide is at least about 18 amino acids in length, about 19 amino acids in length, or about 20 amino acids in length. In exemplary aspects, the peptide is less than about 50 amino acids in length, less than about 40 amino acids, or less than about 30 amino acids, less than about 25 amino acids in length, or less than about 20 amino acids in length. In exemplary aspects, the peptide is about 8 to about 30 amino acids in length or about 10 to about 30 amino acids in length. In exemplary aspects, the peptide is about 10 to about 15 amino acids in length, about 14 to about 20 amino acids in length, about 18 to about 30 amino acids in length, or about 18 to about 26 amino acids in length. In exemplary aspects, the peptide is 11-13, 12-13, 12-14, 13-14, 13-15, 14-15, 14-16, 15-16, 16-18, 16-19, 17-19, 18-19, 20-22, 22-24, 23-24, or 24-25 amino acids in length. In some embodiments, the peptide is a 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer or 30-mer.

According to some embodiments conjugates comprising any of the peptides and analogs described herein conjugated to a moiety for extending half-life or increasing cell penetration. For example, the half-life extending moiety may be a peptide or protein and the conjugate is a fusion protein or chimeric polypeptide. Alternatively, the half-life extending moiety may be a polymer, e.g., a polyethylene glycol. The present disclosures furthermore provide dimers and multimers comprising any of the peptides and analogs described herein.

Any moiety known in the art to facilitate actively or passively or enhance permeability of the peptides into cells may be used for conjugation with the peptide core. Non-limitative examples include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to a preferred embodiment, the hydrophobic moiety is a lipid moiety or an amino acid moiety. The permeability-enhancing moiety may be connected to any position in the peptide moiety, directly or through a spacer or linker, preferably to the amino terminus of the peptide moiety. The hydrophobic moiety may preferably comprise a lipid moiety or an amino acid moiety. According to a specific embodiment the hydrophobic moiety is selected from the group consisting of: phospholipids, steroids, sphingosines, ceramides, octyl-glycine, 2-cyclohexylalanine, benzolylphenylalanine, propionoyl (C₃); butanoyl (C₄); pentanoyl (C₅); caproyl (C₆); heptanoyl (C₇); capryloyl (C₈); nonanoyl (C₉); capryl (C₁₀); undecanoyl (C₁₁); lauroyl (C₁₂); tridecanoyl (C₁₃); myristoyl (C₁₄); pentadecanoyl (C₁₅); palmitoyl (C₁₆); phtanoyl ((CH₃)₄); heptadecanoyl (C₁₆); stearoyl (C₁₈); nonadecanoyl (C₁₉); arachidoyl (C₂₀); heniecosanoyl (C₂₁); behenoyl (C₂₂); trucisanoyl (C₂₃); and lignoceroyl (C₂₄); wherein said hydrophobic moiety is attached to said chimeric polypeptide with amide bonds, sulfhydryls, amines, alcohols, phenolic groups, or carbon-carbon bonds. Other examples of lipidic moieties which may be used include: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DCGS, DPPES, DCPE, DMAP, DMPE, DOGS, DOHME, DPEPC, Pluronic, Tween, BRIJ, plasmalogen, phosphatidylethanolamine, phosphatidylcholine, glycerol-3-ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidylethanolamine, palmitoylhomocystiene, N,N′-Bis (dodecyaminocarbonylmethylene)-N,N′-bis((-N,N,N-trimethylammoniumethyl-aminocarbonylmethylene)ethylenediamine tetraiodide; N,N″-Bis(hexadecylaminocarbonylmethylene)-N,N′,N″-tris((-N,N,N-trimethylammonium-ethylaminocarbonylmethylenediethylenetri amine hexaiodide; N,N′-Bis(dodecylaminocarbonylmethylene)-N,N″-bis((-N,N,N-trimethylammonium ethylaminocarbonylmethylene)cyclohexylene-1,4-diamine tetraiodide; 1,7,7-tetra-((-N,N,N,N-tetramethylammoniumethylamino-carbonylmethylene)-3-hexadecylarninocarbonyl-methylene-1,3,7-triaazaheptane heptaiodide; N,N,N′,N′-tetra((-N,N,N-trimethylammonium-ethylaminocarbonylmethylene)-N′-(1,2-dioleoylglycero-3-phosphoethanolamino carbonylmethylene)diethylenetriamine tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-1-thio-b-D-galactopyranoside, digalactosyldiglyceride, 6-(5-cholesten-3b-yloxy)hexyl-6-amino-6-deoxy-1-thio-b-D-galactopyranoside, 6-(5-cholesten-3b-yloxy)hexyl-6-amino-6-deoxyl-1-thio-a-D-mannopyranoside, 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoyl-phosphatidylethanolamine; 1,2-dioleoyl-sn-glycerol; 1,2-dipalmitoyl-sn-3-succinyl-glycerol; 1,3-dipalmitoyl-2-succinylglycerol, 1-hexadecyl-2-palmitoylglycero-phosphoethanolamine, and palmitoylhomocysteine. 5-Fam is 5-carboxyfluorescein.

The peptides disclosed herein may be conjugated to one or more moieties that cause the conjugate to function as a prodrug. For example, the N-amino acid related moieties described in U.S. Pat. No. 8,969,288 and US Pub. 20160058881 can be conjugated to the peptides disclosed herein and such conjugates are included in this disclosure.

According to some embodiments the peptides may be attached (either covalently or non-covalently) to a penetrating agent. As used herein the phrase “penetrating agent” refers to an agent which enhances translocation of any of the attached peptide across a cell membrane. Typically, peptide based penetrating agents have an amino acid composition containing either a high relative abundance of positively charged amino acids such as lysine or arginine, or have sequences that contain an alternating pattern of polar/charged amino acids and non-polar, hydrophobic amino acids. By way of a non-limiting example, cell penetrating peptide (CPP) sequences may be used in order to enhance intracellular penetration. CPPs may include short and long versions of the protein transduction domain (PTD) of HIV TAT protein, such as for example, YARAAARQARA (SEQ ID NO: 39), YGRKKRR (SEQ ID NO: 40), YGRKKRRQRRR (SEQ ID NO: 41), or RRQRR (SEQ ID NO: 42). However, the disclosure is not so limited, and any suitable penetrating agent may be used, as known by those of skill in the art. Another method of enhancing cell penetration is via N-terminal myristoilation. In this protein modification, a myristoyl group (derived from myristic acid) is covalently attached via an amide bond to the alpha-amino group of an N-terminal amino acid of the peptide.

According to some embodiments the peptide is modified to include a duration enhancing moiety. The duration enhancing moiety can be a water soluble polymer, or a long chain aliphatic group. In some embodiments, a plurality of duration enhancing moieties may be attached to the peptide, in which case each linker to each duration enhancing moiety is independently selected from the linkers described herein.

According to some embodiments the amino terminus of the peptide is modified, e.g. acylated. According to additional embodiments the carboxy terminus is modified, e.g., it may be acylated, conjugated (e.g., with PEG), amidated, reduced or esterified. In accordance with some embodiments, the peptide comprises an acylated amino acid (e.g., a non-coded acylated amino acid (e.g., an amino acid comprising an acyl group which is non-native to a naturally-occurring amino acid)). In accordance with one embodiment, the peptide comprises an acyl group which is attached to the peptide via an ester, thioester, or amide linkage for purposes of prolonging half-life in circulation and/or delaying the onset of and/or extending the duration of action and/or improving resistance to proteases. Acylation can be carried out at any position within the peptide, (e.g., the amino acid at the C-terminus), provided that activity is retained, if not enhanced. The peptide in some embodiments can be acylated at the same amino acid position where a hydrophilic moiety is linked, or at a different amino acid position. The acyl group can be covalently linked directly to an amino acid of the peptide, or indirectly to an amino acid of the peptide via a spacer, wherein the spacer is positioned between the amino acid of the peptide and the acyl group.

In specific aspects, the peptide is modified to comprise an acyl group by direct acylation of an amine, hydroxyl, or thiol of a side chain of an amino acid of the peptide. In this regard, the acylated peptide can comprise the amino acid sequence of any of SEQ ID NO: 1-38, or a modified amino acid sequence thereof comprising one or more of the amino acid modifications described herein.

In some embodiments, the peptide comprises a spacer between the analog and the acyl group. In some embodiments, the peptide is covalently bound to the spacer, which is covalently bound to the acyl group. In some embodiments, the spacer is an amino acid comprising a side chain amine, hydroxyl, or thiol, or a dipeptide or tripeptide comprising an amino acid comprising a side chain amine, hydroxyl, or thiol. The amino acid to which the spacer is attached can be any amino acid (e.g., a singly or doubly α-substituted amino acid) comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol, or a dipeptide or tripeptide comprising an amino acid comprising a side chain amine, hydroxyl, or thiol. When acylation occurs through an amine group of a spacer, the acylation can occur through the alpha amine of the amino acid or a side chain amine. In the instance in which the alpha amine is acylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp, Glu, homoglutamic acid, homocysteic acid, cysteic acid, gamma-glutamic acid. In the instance in which the side chain amine of the amino acid of the spacer is acylated, the amino acid of the spacer is an amino acid comprising a side chain amine. In this instance, it is possible for both the alpha amine and the side chain amine of the amino acid of the spacer to be acylated, such that the peptide is diacylated. Embodiments include such diacylated molecules. When acylation occurs through a hydroxyl group of a spacer, the amino acid or one of the amino acids of the dipeptide or tripeptide can be Ser. When acylation occurs through a thiol group of a spacer, the amino acid or one of the amino acids of the dipeptide or tripeptide can be Cys. In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

In a specific embodiment, the spacer comprises an amino poly(alkyloxy)carboxylate. In this regard, the spacer can comprise, for example, NH₂(CH₂CH₂P)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). In some embodiments, the spacer is a hydrophobic bifunctional spacer. Hydrophobic bifunctional spacers are known in the art. See, e.g., Bioconjugate Techniques, G. T. Hermanson (Academic Press, San Diego, Calif., 1996), which is incorporated by reference in its entirety. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid. In some embodiments, the bifunctional spacer is not a dicarboxylic acid comprising an unbranched, methylene of 1-7 carbon atoms between the carboxylate groups. In some embodiments, the bifunctional spacer is a dicarboxylic acid comprising an unbranched, methylene of 1-7 carbon atoms between the carboxylate groups. The spacer (e g , amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms) in length. In more specific embodiments, the spacer is about 3 to 10 atoms (e.g., 6 to 10 atoms) in length and the acyl group is a C₁₂ to C₁₈ fatty acyl group, e.g., C₁₄ fatty acyl group, C₁₆ fatty acyl group, such that the total length of the spacer and acyl group is 14 to 28 atoms, e.g., about 14, 15, 16, 17, 18, 19, 20, 21, 22, 23, 24, 25, 26, 27, or 28 atoms. In some embodiments, the length of the spacer and acyl group is 17 to 28 (e.g., 19 to 26, 19 to 21) atoms. In accordance with certain foregoing embodiments, the bifunctional spacer can be a synthetic or naturally occurring amino acid (including, but not limited to, any of those described herein) comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer can be the same as or different from the other amino acid(s) of the dipeptide or tripeptide and can be independently selected from the group consisting of: naturally-occurring or coded and/or non-coded or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring or non-coded amino acids selected from the group consisting of: β-alanine ((β-Ala), N-α-methyl-alanine (Me-Ala), aminobutyric acid (Abu), γ-aminobutyric acid (7-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid. In some embodiments, the spacer comprises an overall negative charge, e.g., comprises one or two negative-charged amino acids. In some embodiments, the dipeptide is not any of the dipeptides of general structure A-B, wherein A is selected from the group consisting of Gly, Gln, Ala, Arg, Asp, Asn, Ile, Leu, Val, Phe, and Pro, wherein B is selected from the group consisting of Lys, His, Trp. In some embodiments, the dipeptide spacer is selected from the group consisting of: Ala-Ala, β-Ala-β-Ala, Leu-Leu, Pro-Pro, γ-aminobutyric acid-γ-aminobutyric acid, Glu-Glu, and γ-Glu-γ-Glu.

Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmaceutical Res. “Synthesis of Palmitoyl Derivatives of Insulin and their Biological Activity” Vol. 6, No: 2 pp. 171-176 (1989). The acyl group of the acylated amino acid can be of any size, e.g., any length carbon chain, and can be linear or branched. In some specific embodiments, the acyl group is a C₄ to C₃₀ fatty acid. For example, the acyl group can be any of a C₄ fatty acid, C₆ fatty acid, C₈ fatty acid, C₁₀ fatty acid, C₁₂ fatty acid, C₁₄ fatty acid, C₁₆ fatty acid, C₁₈ fatty acid, C₂₀ fatty acid, C₂₂ fatty acid, C₂₄ fatty acid, C₂₆ fatty acid, C₂₈ fatty acid, or a C₃₀ fatty acid. In some embodiments, the acyl group is a C₈ to C₂₀ fatty acid, e.g., a C₁₄ fatty acid or a C₁₆ fatty acid. In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid. In some embodiments, the peptide comprises an acylated amino acid by acylation of a long chain alkane on the peptide. In specific aspects, the long chain alkane comprises an amine, hydroxyl, or thiol group (e.g., octadecylamine, tetradecanol, and hexadecanethiol) which reacts with a carboxyl group, or activated form thereof, of the peptide. The carboxyl group, or activated form thereof, of the peptide can be part of a side chain of an amino acid (e.g., glutamic acid, aspartic acid) of the peptide or can be part of the analog backbone. In certain embodiments, the peptide is modified to comprise an acyl group by acylation of the long chain alkane by a spacer which is attached to the peptide. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers.

As used herein, the term “activated form” of a carboxyl group refers to a carboxyl group with the general formula R(C═O)X, wherein X is a leaving group and R is the peptide or the spacer. For example, activated forms of a carboxyl groups may include, but are not limited to, acyl chlorides, anhydrides, and esters. In some embodiments, the activated carboxyl group is an ester with a N-hydroxysuccinimide ester (NHS) leaving group.

With regard to these aspects, in which a long chain alkane is acylated by the peptide or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane.

Also, in some embodiments, an amine, hydroxyl, or thiol group of the peptide is acylated with a cholesterol acid. In a specific embodiment, the peptide is linked to the cholesterol acid through an alkylated des-amino Cys spacer, i.e., an alkylated 3-mercaptopropionic acid spacer. The alkylated des-amino Cys spacer can be, for example, a des-amino-Cys spacer comprising a dodecaethylene glycol moiety.

The peptides described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In this regard, the acylated peptide can be any of SEQ ID NOs: 1-38, including any of the modifications described herein, in which at least one of the amino acids comprises an acyl group and at least one of the amino acids is covalently bonded to a hydrophilic moiety (e.g., PEG). In some embodiments, the acyl group is attached via a spacer comprising Cys, Lys, Orn, homo-Cys, or Ac-Phe, and the hydrophilic moiety is incorporated at a Cys residue or at the C-terminus.

Alternatively, the peptides can comprise a spacer, wherein the spacer is both acylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In accordance with some embodiments, the peptide comprises an alkylated amino acid (e.g., a non-coded alkylated amino acid (e.g., an amino acid comprising an alkyl group which is non-native to a naturally-occurring amino acid)). Alkylation can be carried out at any positions within the peptides, including any of the positions described herein as a site for acylation, including but not limited to, any of amino acid positions, at a position within a C-terminal extension, or at the C-terminus, provided that the biological activity is retained. The alkyl group can be covalently linked directly to an amino acid of the peptides, or indirectly to an amino acid of the peptides via a spacer, wherein the spacer is positioned between the amino acid of the peptides and the alkyl group. The peptides may be alkylated at the same amino acid position where a hydrophilic moiety is linked, or at a different amino acid position. In specific aspects, the peptides may be modified to comprise an alkyl group by direct alkylation of an amine, hydroxyl, or thiol of a side chain of an amino acid of the peptides. In this regard, the alkylated peptides can comprise an amino acid sequence with at least one of the amino acids modified to any amino acid comprising a side chain amine, hydroxyl, or thiol. In yet other embodiments, the amino acid comprising a side chain amine, hydroxyl, or thiol is a disubstituted amino acid. In some embodiments, the alkylated peptide comprises a spacer between the peptide and the alkyl group. In some embodiments, the peptide is covalently bound to the spacer, which is covalently bound to the alkyl group. In some exemplary embodiments, the peptide is modified to comprise an alkyl group by alkylation of an amine, hydroxyl, or thiol of a spacer, which spacer is attached to a side chain of an amino acid. The amino acid to which the spacer is attached can be any amino acid comprising a moiety which permits linkage to the spacer. For example, an amino acid comprising a side chain NH₂, —OH, or —COOH (e.g., Lys, Orn, Ser, Asp, or Glu) is suitable. In some embodiments, the spacer is an amino acid comprising a side chain amine, hydroxyl, or thiol or a dipeptide or tripeptide comprising an amino acid comprising a side chain amine, hydroxyl, or thiol. When alkylation occurs through an amine group of a spacer, the alkylation can occur through the alpha amine of an amino acid or a side chain amine In the instance in which the alpha amine is alkylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp and Glu, provided that the alkylation occurs on the alpha amine of the acidic residue. In the instance in which the side chain amine of the amino acid of the spacer is alkylated, the amino acid of the spacer is an amino acid comprising a side chain amine, e.g., an amino acid of Formula I (e.g., Lys or Orn). In this instance, it is possible for both the alpha amine and the side chain amine of the amino acid of the spacer to be alkylated, such that the peptide is dialkylated. Embodiments include such dialkylated molecules. When alkylation occurs through a hydroxyl group of a spacer, the amino acid can be Ser. When alkylation occurs through a thiol group of spacer, the amino acid can be Cys. In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). Suitable hydrophobic bifunctional spacers comprising a carboxylate and a hydroxyl group or a thiol group are known in the art and include, for example, 8-hydroxyoctanoic acid and 8-mercaptooctanoic acid. The spacer (e g , amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms)) in length. In more specific embodiments, the spacer is about 3 to 10 atoms (e.g., 6 to 10 atoms) in length and the alkyl is a C₁₂ to C₁₈ alkyl group, e.g., C₁₄ alkyl group, C₁₆ alkyl group, such that the total length of the spacer and alkyl group is 14 to 28 atoms, e.g., about 14, 15, 16, 17, 18, 19, 20, 21, 22, 23, 24, 25, 26, 27, or 28 atoms. In some embodiments, the length of the spacer and alkyl is 17 to 28 (e.g., 19 to 26, 19 to 21) atoms. In accordance with certain foregoing embodiments, the bifunctional spacer can be a synthetic or non-naturally occurring or non-coded amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. The dipeptide or tripeptide spacer can be composed of naturally-occurring or coded and/or non-coded or non-naturally occurring amino acids, including, for example, any of the amino acids taught herein. In some embodiments, the spacer comprises an overall negative charge, e.g., comprises one or two negative-charged amino acids. In some embodiments, the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu. Suitable methods of peptide alkylation via amines, hydroxyls, and thiols are known in the art. For example, a Williamson ether synthesis can be used to form an ether linkage between a hydroxyl group of the peptides and the alkyl group. Also, a nucleophilic substitution reaction of the peptide with an alkyl halide can result in any of an ether, thioether, or amino linkage. The alkyl group of the alkylated peptides can be of any size, e.g., any length carbon chain, and can be linear or branched. In some embodiments, the alkyl group is a C₄ to C₃₀ alkyl. For example, the alkyl group can be any of a C₄ alkyl, C₆ alkyl, C₈ alkyl, C₁₀ alkyl, C₁₂ alkyl, C₁₄ alkyl, C₁₆ alkyl, C₁₈ alkyl, C₂₀ alkyl, C₂₂ alkyl, C₂₄ alkyl, C₂₆ alkyl, C₂₈ alkyl, or a C₃₀ alkyl. In some embodiments, the alkyl group is a C₈ to C₂₀ alkyl, e.g., a C₁₄ alkyl or a C₁₆ alkyl. In some embodiments of the disclosure, the peptide comprises an alkylated amino acid by reacting a nucleophilic, long chain alkane with the peptide, wherein the peptide comprises a leaving group suitable for nucleophilic substitution. In specific aspects, the nucleophilic group of the long chain alkane comprises an amine, hydroxyl, or thiol group (e.g., octadecylamine, tetradecanol, and hexadecanethiol). The leaving group of the peptide can be part of a side chain of an amino acid or can be part of the peptide backbone. Suitable leaving groups include, for example, N-hydroxysuccinimide, halogens, and sulfonate esters. In certain embodiments, the peptide is modified to comprise an alkyl group by reacting the nucleophilic, long chain alkane with a spacer which is attached to the peptide, wherein the spacer comprises the leaving group. In specific aspects, the long chain alkane comprises an amine, hydroxyl, or thiol group. In certain embodiments, the spacer comprising the leaving group can be any spacer discussed herein, e g , amino acids, dipeptides, tripeptides, hydrophilic bifunctional spacers and hydrophobic bifunctional spacers further comprising a suitable leaving group. With regard to these aspects of the disclosure, in which a long chain alkane is alkylated by the peptides or the spacer, the long chain alkane may be of any size and can comprise any length of carbon chain. The long chain alkane can be linear or branched. In certain aspects, the long chain alkane is a C₄ to C₃₀ alkane For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane. Also, in some embodiments, alkylation can occur between the peptides and a cholesterol moiety. For example, the hydroxyl group of cholesterol can displace a leaving group on the long chain alkane to form a cholesterol-peptides product. The alkylated peptides described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. Alternatively, the alkylated peptides can comprise a spacer, wherein the spacer is both alkylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In some embodiments, the peptide comprises at position 1 or 2, or at both positions 1 and 2, an amino acid which achieves resistance of the peptides to peptidase cleavage. In some embodiments, the peptide comprises at position 1 an amino acid selected from the group consisting of: D-histidine, desaminohistidine, hydroxyl-histidine, acetyl-histidine, homo-histidine, N-methyl histidine, alpha-methyl histidine, imidazole acetic acid, or alpha, alpha-dimethyl imidazole acetic acid (DMIA). In some embodiments, the peptide comprises at position 2 an amino acid selected from the group consisting of: D-serine, D-alanine, valine, glycine, N-methyl serine, N-methyl alanine, or alpha, aminoisobutyric acid. In some embodiments, the peptide comprises at position 2 an amino acid which achieves resistance of the peptide to peptidases and the amino acid which achieves resistance of the peptide to peptidases is not D-serine. In some embodiments, this covalent bond is an intramolecular bridge other than a lactam bridge. For example, suitable covalent bonding methods include any one or more of olefin metathesis, lanthionine-based cyclization, disulfide bridge or modified sulfur-containing bridge formation, the use of α,ω-diaminoalkane tethers, the formation of metal-atom bridges, and other means of peptide cyclization.

In some embodiments, the peptide is modified by amino acid substitutions and/or additions that introduce a charged amino acid into the C-terminal portion of the analog. In some embodiments, such modifications enhance stability and solubility. As used herein the term “charged amino acid” or “charged residue” refers to an amino acid that comprises a side chain that is negative-charged (i.e., de-protonated) or positive-charged (i.e., protonated) in aqueous solution at physiological pH. In some aspects, these amino acid substitutions and/or additions that introduce a charged amino acid modifications may be at a C-terminal position. In some embodiments, one, two or three (and in some instances, more than three) charged amino acids may be introduced at the C-terminal position. In exemplary embodiments, one, two or all of the charged amino acids may be negative-charged. The negative-charged amino acid in some embodiments is aspartic acid, glutamic acid, cysteic acid, homocysteic acid, or homoglutamic acid. In some aspects, these modifications increase solubility.

In accordance with some embodiments, the peptides disclosed herein may be modified by truncation of the C-terminus by one or two amino acid residues. In this regard, the peptides can comprise the sequences (SEQ ID NO: 1-38), optionally with any of the additional modifications described herein.

In some embodiments, the peptide comprises a modified SEQ ID NO: 1-38 in which the carboxylic acid of the C-terminal amino acid is replaced with a charge-neutral group, such as an amide or ester. Accordingly, in some embodiments, the peptide is an amidated peptide, such that the C-terminal residue comprises an amide in place of the alpha carboxylate of an amino acid. As used herein a general reference to a peptide or analog is intended to encompass peptides that have a modified amino terminus, a modified carboxy terminus, or modifications of both amino and carboxy termini For example, an amino acid chain composing an amide group in place of the terminal carboxylic acid is intended to be encompassed by an amino acid sequence designating the standard amino acids.

In accordance with some embodiments, the peptides disclosed herein may be modified by conjugation on at least one amino acid residue. In this regard, the peptides can comprise the sequences (SEQ ID NO: 1-38), optionally with any of the additional conjugations described herein.

The disclosure further provides conjugates comprising one or more of the peptides described herein conjugated to a heterologous moiety. As used herein, the term “heterologous moiety” is synonymous with the term “conjugate moiety” and refers to any molecule (chemical or biochemical, naturally-occurring or non-coded) which is different from the peptides described herein. Exemplary conjugate moieties that can be linked to any of the analogs described herein include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising a peptide and a plasma protein, wherein the plasma protein is selected from the group consisting of albumin, transferin, fibrinogen and globulins. In some embodiments the plasma protein moiety of the conjugate is albumin or transferin.

The conjugate in some embodiments comprises one or more of the peptides described herein and one or more of: a different peptide (which is distinct from the peptides described herein), a polypeptide, a nucleic acid molecule, an antibody or fragment thereof, a polymer, a quantum dot, a small molecule, a toxin, a diagnostic agent, a carbohydrate, an amino acid. In some embodiments, the heterologous moiety is a polymer. In some embodiments, the polymer is selected from the group consisting of: polyamides, polyvarhonateN, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene. In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion. In some aspects, the polymer is a bioadhesive polymer, such as a bioerodible hydrogel described by H. S. Sawhney, C. P. Pathak and J. A. Hubbell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some embodiments, the polymer is a water-soluble polymer or a hydrophilic polymer. Hydrophilic polymers are further described herein under “Hydrophilic Moieties.” Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof. In specific embodiments, the polymer is a polyalkylene glycol, including, for example, polyethylene glycol (PEG).

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerois), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is attached via non-covalent or covalent bonding to the peptide of the present disclosure. In certain aspects, the heterologous moiety is attached to the peptide of the present disclosure via a linker. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other. The peptide in some embodiments is linked to conjugate moieties via direct covalent linkage by reacting targeted amino acid residues of the analog with an organic derivatizing agent that is capable of reacting with selected side chains or the N- or C-terminal residues of these targeted amino acids. Reactive groups on the analog or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art. Alternatively, the conjugate moieties can be linked to the analog indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier. Cysteinyl residues are most commonly reacted with α-haloacetates (and corresponding amines), such as chloroacetic acid, chloroacetamide to give carboxymethyl or carboxyamidomethyl derivatives. Cysteinyl residues also may be derivatized by reaction with bromotrifluoroacetone, alpha-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues may be derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino-terminal residues may be reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Carboxyl side groups (aspartyl or glutamyl) may be selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions. Other modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)), deamidation of asparagine or glutamine, acetylation of the N-terminal amine, and/or amidation or esterification of the C-terminal carboxylic acid group. Another type of covalent modification involves chemically or enzymatically coupling glycosides to the peptide. Sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981). In some embodiments, the peptide is conjugated to a heterologous moiety via covalent linkage between a side chain of an amino acid of the peptides and the heterologous moiety. In some aspects, the amino acid covalently linked to a heterologous moiety (e.g., the amino acid comprising a heterologous moiety) is a Cys, Lys, Orn, homo-Cys, or Ac-Phe, and the side chain of the amino acid is covalently bonded to a heterologous moiety. In some embodiments, the conjugate comprises a linker that joins the peptide to the heterologous moiety. In some aspects, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms may be all carbon atoms. In some embodiments, the chain atoms in the backbone of the linker may be selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. If the linker is a covalent bond or a peptidyl bond and the conjugate is a polypeptide, the entire conjugate can be a fusion protein. Such peptidyl linkers may be any length. Exemplary linkers may be from about 1 to 50 amino acids in length, 5 to 50, 3 to 5, 5 to 10, 5 to 15, or 10 to 30 amino acids in length. Such fusion proteins may alternatively be produced by recombinant genetic engineering methods known to one of ordinary skill in the art.

As noted above, in some embodiments, the peptides may be conjugated, e.g., fused to an immunoglobulin or portion thereof (e.g., variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC). For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CH2 domain of a human IgG Fc region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In related embodiments, the Fc region may comprise one or more native or modified constant regions from an immunoglobulin heavy chain, other than CH1, for example, the CH2 and CH3 regions of IgG and IgA, or the CH3 and CH4 regions of IgE. Suitable conjugate moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the CH3 domain Some conjugate moieties may or may not include FcγR binding site(s). FcγR are responsible for ADCC and CDC. Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005) Amino acid residues involved in IgE receptor binding are described in Sayers et al. (J Biol Chem. 279(34):35320-5, 2004). Amino acid modifications may be made to the Fc region of an immunoglobulin. Such variant Fc regions comprise at least one amino acid modification in the CH3 domain of the Fc region (residues 342-447) and/or at least one amino acid modification in the CH2 domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591) Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety. In certain embodiments, a peptide described herein is inserted into a loop region within the immunoglobulin molecule. In other embodiments, a peptide described herein replaces one or more amino acids of a loop region within the immunoglobulin molecule.

The peptides described herein can be further modified to improve its solubility and stability in aqueous solutions at physiological pH, while retaining the biological activity. Hydrophilic moieties such as PEG groups can be attached to the analogs under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the analog (e.g., an acid, aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the analog by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995). In specific aspects, an amino acid residue of the peptides having a thiol is modified with a hydrophilic moiety such as PEG. In some embodiments, the thiol is modified with maleimide-activated PEG in a Michael addition reaction to result in a PEGylated analog comprising a thioether linkage. In some embodiments, the thiol is modified with a haloacetyl-activated PEG in a nucleophilic substitution reaction to result in a PEGylated analog comprising a thioether linkage. Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C₁-C₁₀) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by al-6 linkages. Dextran is available in many molecular weight ranges, e.g., about 1 kD to about 100 kD, or from about 5, 10, 15 or 20 kD to about 30, 40, 50, 60, 70, 80 or 90 kD. Linear or branched polymers are contemplated. Resulting preparations of conjugates may be essentially monodisperse or polydisperse, and may have about 0.5, 1, 1.2, 1.5 or 2 polymer moieties per analog.

In some embodiments, the peptide is conjugated to a hydrophilic moiety via covalent linkage between a side chain of an amino acid of the peptide and the hydrophilic moiety. In some embodiments, the peptide is conjugated to a hydrophilic moiety via the side chain of an amino acid, a position within a C-terminal extension, or the C-terminal amino acid, or a combination of these positions. In some aspects, the amino acid covalently linked to a hydrophilic moiety (e.g., the amino acid comprising a hydrophilic moiety) is a Cys, Lys, Orn, homo-Cys, or Ac-Phe, and the side chain of the amino acid is covalently bonded to a hydrophilic moiety (e.g., PEG). In some embodiments, the conjugate of the present disclosure comprises the peptide fused to an accessory analog which is capable of forming an extended conformation similar to chemical PEG (e.g., a recombinant PEG (rPEG) molecule), such as those described in International Patent Application Publication No. WO2009/023270 and U.S. Patent Application Publication No. US20080286808. The rPEG molecule in some aspects is a polypeptide comprising one or more of glycine, serine, glutamic acid, aspartic acid, alanine, or proline. In some aspects, the rPEG is a homopolymer, e.g., poly-glycine, poly-serine, poly-glutamic acid, poly-aspartic acid, poly-alanine, or poly-proline. In other embodiments, the rPEG comprises two types of amino acids repeated, e.g., poly(Gly-Ser), poly(Gly-Glu), poly(Gly-Ala), poly(Gly-Asp), poly(Gly-Pro), poly(Ser-Glu), etc. In some aspects, the rPEG comprises three different types of amino acids, e.g., poly(Gly-Ser-Glu). In specific aspects, the rPEG increases the half-life of the peptide. In some aspects, the rPEG comprises a net positive or net negative charge. The rPEG in some aspects lacks secondary structure. In some embodiments, the rPEG is greater than or equal to 10 amino acids in length and in some embodiments is about 40 to about 50 amino acids in length. The accessory peptide in some aspects is fused to the N- or C-terminus of the peptide of the present disclosure through a peptide bond or a proteinase cleavage site, or is inserted into the loops of the peptide of the present disclosure. The rPEG in some aspects comprises an affinity tag or is linked to a PEG that is greater than 5 kDa. In some embodiments, the rPEG confers the peptide of the present disclosure with an increased hydrodynamic radius, serum half-life, protease resistance, or solubility and in some aspects confers the analog with decreased immunogenicity.

The peptides comprising the sequences (SEQ ID NO: 1-38), optionally with any of the conjugations described herein are contemplated as an embodiment.

The disclosure further provides multimers or dimers of the peptides disclosed herein, including homo- or hetero-multimers or homo- or hetero-dimers. Two or more of the analogs can be linked together using standard linking agents and procedures known to those skilled in the art. For example, dimers can be formed between two peptides through the use of bifunctional thiol crosslinkers and bi-functional amine crosslinkers, particularly for the analogs that have been substituted with cysteine, lysine ornithine, homocysteine or acetyl phenylalanine residues. The dimer can be a homodimer or alternatively can be a heterodimer. In certain embodiments, the linker connecting the two (or more) analogs is PEG, e.g., a 5 kDa PEG, 20 kDa PEG. In some embodiments, the linker is a disulfide bond. For example, each monomer of the dimer may comprise a Cys residue (e.g., a terminal or internally positioned Cys) and the sulfur atom of each Cys residue participates in the formation of the disulfide bond. In some aspects, the monomers may be connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer may be attached together in a “tail-to-tail” orientation in which the C-terminal amino acids of each monomer may be attached together.

Peptides disclosed herein may be made in a variety of ways. Suitable methods of de novo synthesizing peptides are described in, for example, Merrifield, J. Am. Chem. Soc, 85, 2149 (1963); Davis et al., Biochem. Intl., 10, 394-414 (1985); Larsen et al., J. Am. Chem. Soc, 115, 6247 (1993); Smith et al., J. Peptide Protein Res., 44, 183 (1994); O′Donnell et al., J. Am. Chem. Soc, 118, 6070 (1996); Stewart and Young, Solid Phase Peptide Synthesis, Freeman (1969); Finn et al., The Proteins, 3 ed., vol. 2, pp. 105-253 (1976); Erickson et al., The Proteins, 3^{rd} ed., vol. 2, pp. 257-527 (1976); and Chan et al., Fmoc Solid Phase Peptide Synthesis, Oxford University Press, Oxford, United Kingdom, 2005. The disclosure contemplates synthetic peptides. Methods of making the peptides are themselves embodiments of the invention.

Alternatively, the peptide can be expressed recombinantly by introducing a nucleic acid that comprises or consists of a nucleotide sequence encoding a peptide into host cells, which may be cultured to express the encoded peptide using standard recombinant methods. See, for instance, Sambrook et al., Molecular Cloning: A Laboratory Manual. 3rd ed., Cold Spring Harbor Press, Cold Spring Harbor, N.Y. 2001; and Ausubel et al., Current Protocols in Molecular Biology, Greene Publishing Associates and John Wiley & Sons, N.Y., 1994.Such peptides may be purified from the culture media or cell pellets. Exemplary nucleic acids include deoxyribonucleic acid (DNA) and ribonucleic acids (RNA).

In some embodiments, the peptides of the disclosure can be isolated. In some embodiments, the peptides of the disclosure may be purified. It is recognized that “purity” is a relative term, and not to be necessarily construed as absolute purity or absolute enrichment or absolute selection. In some aspects, the purity is at least or about 50%, is at least or about 60%, at least or about 70%, at least or about 80%, or at least or about 90% (e.g., at least or about 91%, at least or about 92%, at least or about 93%, at least or about 94%, at least or about 95%, at least or about 96%, at least or about 97%, at least or about 98%, at least or about 99% or is approximately 100%.

In some embodiments, the peptides described herein can be commercially synthesized by companies, such as Genscript (Piscataway, NJ), New England Peptide (Gardner, MA), and CPC Scientific (Sunnyvale, CA), Peptide Technologies Corp. (Gaithersburg, Md.), and Multiple Peptide Systems (San Diego, Calif.). In this respect, the peptides can be synthetic, recombinant, isolated, and/or purified.

The present disclosure also includes, as an additional embodiment, compositions that comprise mixture of two or more peptides or peptide analogs described herein (or conjugates, nucleic acids, expression vectors, etc.), optionally further including an excipient or carrier.

The peptides of the present disclosure can be provided in accordance with one embodiment as part of a kit. Accordingly, in some embodiments, a kit for administering a peptide, to a patient in need thereof is provided wherein the kit comprises a peptide as described herein.

In one embodiment the kit is provided with a device for adrninistring the composition to a patient, e.g., syringe needle, pen device, jet injector or another needle-free injector. The kit may alternatively or in addition include one or more containers, e.g., vials, tubes, bottles, single or multi-chambered pre-filled syringes, cartridges, infusion pumps (external or implantable), jet injectors, pre-filled pen devices and the like, optionally containing the peptide in a lyophilized form or in an aqueous solution. The kits in some embodiments comprise instructions for use. In accordance with one embodiment the device of the kit is an aerosol dispensing device, wherein the composition is prepackaged within the aerosol device. In another embodiment the kit comprises a syringe and a needle, and in one embodiment the sterile composition is prepackaged within the syringe.

A further embodiment includes a process of treating a disease comprising one or more of prescribing, selling or advertising to sell, purchasing, instructing to self-administer, or administering a peptide described herein, wherein the peptide has been approved by a regulatory agency for the treatment of a condition, to a subject in need of treatment.

A further embodiment includes a method of supplying a peptide for treating a disease, said method comprises reimbursing a physician, a formulary, a patient or an insurance company for the sale of said peptide.

### Definitions

The terms “peptide” refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified peptides. A peptide may be monomeric or polymeric. In certain embodiments, “peptides” are chains of amino acids whose alpha carbons may be linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

The term “therapeutic peptide” refers to peptides or fragments or variants thereof, having one or more therapeutic and/or biological activities.

The term “analog” as used herein describes a peptide comprising one or more amino acid modifications, such as but not limited to substitution and/or one or more deletion and/or one or more addition of any one of the amino acid residues for any natural or unnatural amino acid, synthetic amino acids or peptidomimetics and/or the attachment of a side chain to any one of the natural or unnatural amino acids, synthetic amino acids or peptidomimetics at any available position. The addition or deletion of amino acid residues can take place at the N-terminal of the peptide and/or at the C-terminal of the peptide.

In some embodiments, the analog has 1, 2, 3, 4, or 5 such modifications. In some embodiments, the analog retains biological activity of the original peptide. In some embodiments, the analog is a competitive or non-competitive inhibitor of the original peptide.

Peptide sequences are indicated using standard one- or three-letter abbreviations. Unless otherwise indicated, peptide sequences have their amino termini at the left and their carboxy termini at the right, A particular section of a peptide can be designated by amino acid residue number such as amino acids 3 to 6, or by the actual residue at that site such as Met3 to Gly6. A particular peptide sequence also can be described by explaining how it differs from a reference sequence.

When used herein the term “natural amino acid” is an amino acid (with the usual three letter codes & one letter codes in parenthesis) selected from the group consisting of: Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (His & H), lysine (Lys & K), arginine (Arg & R), glutamine (Gin & Q), asparagine (Asn & N), glutamic acid (Glu & E), aspartic acid (Asp & D), serine (Ser & S) and threonine (Thr & T). If anywhere herein, reference is made to a peptide, analog or derivative or peptides comprising or not comprising G, P, A, V, L, I, M, C, F, Y, H, K, R, Q, N, E, D, S or T, without specifying further, amino acids are meant. If not otherwise indicated amino acids indicated with a single letter code in CAPITAL letters indicate the L-isoform, if however, the amino acid is indicated with a lower case letter, this amino acid is used/applied as it's D-form. Such D-forms and other non-conservative amino acid substitutions previously defined are included in a definition of unnatural amino acids.

If, due to typing errors, there are deviations from the commonly used codes, the commonly used codes apply. The amino acids present in the peptides are, preferably, amino acids which can be coded for by a nucleic acid. As is apparent from the above examples, amino acid residues may be identified by their full name, their one-letter code, and/or their three-letter code. These three ways are fully equivalent.

A “non-conservative amino acid substitution” also refers to the substitution of a member of one of these classes for a member from another class. In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included. It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Other amino acid substitutions are set forth in Table 3.

TABLE 3

| Original | | Preferred |
|---|---|---|
| Residues | Substitutions | Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Nle | Leu |
| Leu | Norleucine, Ile, Val, Met, Ala, Phe | Ile |
| Lys | Arg, Gln, Asn, 1,4-Diamino-butyric Acid | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Nle | Leu |

As used herein the term “charged amino acid” or “charged residue” refers to an amino acid that comprises a side chain that is negative-charged (i.e., de-protonated) or positive-charged (i.e., protonated) in aqueous solution at physiological pH. For example, negative-charged amino acids include aspartic acid, glutamic acid, cysteic acid, homocysteic acid, and homoglutamic acid, whereas positive-charged amino acids include arginine, lysine and histidine. Charged amino acids include the charged amino acids among the 20 coded amino acids, as well as atypical or non-naturally occurring or non-coded amino acids.

As used herein the term “acidic amino acid” refers to an amino acid that comprises a second acidic moiety (other than the carboxylic acid of the amino acid), including for example, a carboxylic acid or sulfonic acid group.

As used herein, the term “acylated amino acid” refers to an amino acid comprising an acyl group which is non-native to a naturally-occurring amino acid, regardless of the means by which it is produced (e.g. acylation prior to incorporating the amino acid into a peptide, or acylation after incorporation into a peptide).

As used herein the term “alkylated amino acid” refers to an amino acid comprising an alkyl group which is non-native to a naturally-occurring amino acid, regardless of the means by which it is produced. Accordingly, the acylated amino acids and alkylated amino acids of the present disclosures are non-coded amino acids.

A skilled artisan will be able to determine active variants of peptides as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar peptides. In further embodiments, even areas that may be important for biological activity or for structure may be subject to conservative amino acid substitutions without destroying the biological activity or without adversely affecting the peptide structure.

Additionally, one skilled in the art can review structure-function studies identifying residues in similar peptides that are important for activity or structure. In view of such a comparison, the skilled artisan can predict the importance of amino acid residues in a peptide that correspond to amino acid residues important for activity or structure in similar peptides. One skilled in the art may opt for chemically similar amino acid substitutions for such predicted important amino acid residues.

One skilled in the art can also analyze the three-dimensional structure and amino acid sequence in relation to that structure in similar peptides. In view of such information, one skilled in the art may predict the alignment of amino acid residues of a peptide with respect to its three-dimensional structure. In certain embodiments, one skilled in the art may choose to not make radical changes to amino acid residues predicted to be on the surface of the peptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “derivative” as used herein means a chemically modified peptide, in which one or more side chains have been covalently attached to the peptide. The term “side chain” may also be referred to as a “substituent”. A derivative comprising such side chains will thus be “derivatized” peptide or “derivatized” analog. The term may also refer to peptides containing one or more chemical moieties not normally a part of the peptide molecule such as esters and amides of free carboxy groups, acyl and alkyl derivatives of free amino groups, phospho esters and ethers of free hydroxy groups. Such modifications may be introduced into the molecule by reacting targeted amino acid residues of the peptide with an organic derivatizing agent that is capable of reacting with selected side chains or terminal residues. Preferred chemical derivatives include peptides that have been phosphorylated, C-termini amidated or N-termini acetylated. The term may also refer to peptides as used herein which may be prepared from the functional groups which occur as side chains on the residues or the N- or C-terminal groups, by means known in the art, and are included herein as long as they remain pharmaceutically acceptable, i.e., they do not destroy the activity of the peptide, do not confer toxic properties on compositions containing it and do not adversely affect the antigenic properties thereof. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

A modified amino acid residue is an amino acid residue in which any group or bond was modified by deletion, addition, or replacement with a different group or bond, as long as the functionality of the amino acid residue is preserved or if functionality changed (for example replacement of tyrosine with substituted phenylalanine) as long as the modification did not impair the activity of the peptide containing the modified residue.

The term “substituent” or “side chain” as used herein means any suitable moiety bonded, in particular covalently bonded, to an amino acid residue, in particular to any available position on an amino acid residue. Typically, the suitable moiety is a chemical moiety.

The term “fatty acid” refers to aliphatic monocarboxylic acids having from 4 to 28 carbon atoms, it is preferably un-branched, and it may be saturated or unsaturated. In the present disclosure fatty acids comprising 10 to 16 amino acids are preferred.

The term “fatty diacid” refers to fatty acids as defined above but with an additional carboxylic acid group in the omega position. Thus, fatty diacids are dicarboxylic acids. In the present disclosure fatty acids comprising 14 to 20 amino acids are preferred.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence.

Exemplary computer programs which can be used to determine degrees of identity or homology between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. (Id). In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance.

A “pharmaceutical composition” refers to a composition suitable for pharmaceutical use in an animal or human. A pharmaceutical composition comprises a pharmacologically and/or therapeutically effective amount of an active agent and a pharmaceutically acceptable excipient or carrier. Pharmaceutical compositions and methods for their preparation will be readily apparent to those skilled in the art. Such compositions and methods for their preparation may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration. The term also encompasses any of the agents listed in the US Pharmacopeia for use in animals, including humans. Suitable pharmaceutical carriers and formulations are described in Remington's Pharmaceutical Sciences, 21st Ed. 2005, Mack Publishing Co, Easton.

“Pharmaceutically acceptable carrier” or “pharmaceutically acceptable excipient” refers to compositions that do not produce adverse, allergic, or other untoward reactions when administered to an animal or a human As used herein, “pharmaceutically acceptable carrier” or “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, the excipients will include isotonic agents, for example, sugars, polyalcohols such as mannitol, sorbitol, or sodium chloride in the composition. Additional examples of pharmaceutically acceptable excipients are wetting agents or minor amounts of auxiliary substances such as wetting or emulsifying agents, preservatives or buffers, which enhance the shelf life or effectiveness of the peptide.

As used herein the term “pharmaceutically acceptable salt” refers to salts of peptides that retain the biological activity of the parent peptide, and which are not biologically or otherwise undesirable. Many of the peptides disclosed herein are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

It may be convenient or desirable to prepare, purify, and/or handle a corresponding solvate of the peptide. The term “solvate” is used herein in the conventional sense to refer to a complex of solute (e.g., peptide, salt of peptide) and solvent. If the solvent is water, the solvate may be conveniently referred to as a hydrate, for example, a mono-hydrate, a di-hydrate, a tri-hydrate, etc. Unless otherwise specified, a reference to a particular peptide also includes solvate and hydrate forms thereof.

The “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a peptide of Formula I, and a guest (or co-former) molecule or molecules.

As used herein, a “therapeutically effective amount” of a peptide that when provided to a subject in accordance with the disclosed and claimed methods beneficially affects biological activities, symptoms, disease development, or disease progression. In the context of fibrotic conditions in general, evidence of efficacy includes a reduction in inflammatory markers, reduction in inflammatory cells (in the blood or localized), and rejection of collagen (soluble or localized). In the context of IPF, one or more of improved breathing capacity, reduced feelings of shortness of breath, reduced cough, reduced swelling, reduction of lung scarring, slowing the progression of lung scarring, slowing the reduction of breathing capacity, reducing or delaying the need for oxygen therapy are indicia of therapeutic efficacy. On a cellular level, evidence of reduced epithelial injury and/or reduced collagen deposition are evidence of therapeutic efficacy. On an individual patient basis, evidence of improved score in a pulmony function test or walk test is evidence of therapeutic efficacy.

The terms “treat”, “treating” and “treatment” refer refers to an approach for obtaining beneficial or desired clinical results. Further, references herein to “treatment” include references to curative, palliative and prophylactic treatment. The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology. It will be understood that therapeutic agents typically are not effective in 100% of affected/treated individuals, but therapeutic efficacy can be demonstrated with a statistically significant improvement in one or more disease parameters in a population study (e.g., a clinical trial). Evidence of therapeutic efficacy also can be generated in pre-clinical animal models of disease. Similarly, palliative efficacy can be evinced in a pre-clinical or clinical study in which a treatment group shows a favorable course of disease (e.g., slower progression, better quality-of-life), measured by one or more disease-relevant parameters, compared to a control group.

In the context of combination therapy, “synergy” refers to an effect of two agents or drugs that is greater or stronger than an additive effect. Amounts of drugs that achieve such an effect are synergistically effective amounts. Synergy can be measured in absolute amounts (e.g., at least 10%, at least 20%, at least 30%, at least 40%, at least 50%, at least 60%, at least 70%, at least 80%, at least 90%, at least 100%, at least 150%, at least 200%, at least 300%, at least 500% greater than additive effect. Synergy also can be measured statistically. Synergy can exist with respect to one therapeutically relevant property or multiple properties.

The term “improving cell survival” refers to an increase in the number of cells that survive a given condition, as compared to a control, e.g., the number of cells that would survive the same conditions in the absence of treatment. Conditions can be in vitro, in vivo, ex vivo, or in situ. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition.

For clarity, the term “instructing” is meant to include information on a label approved by a regulatory agency, in addition to its commonly understood definition.

### Gene Therapy

In an embodiment, the peptides may be administered as their nucleotide equivalents via gene therapy methods. The term “nucleotide equivalents” includes any nucleic acid which includes a nucleotide sequence that encodes a peptide. For example, the invention includes polynucleotides that comprise or conist of a nucleotide sequence that encodes a peptide described herein. The invention also includes vectors, including exression vectors, that comprise a nucleoide sequence that encodes a peptide described herein. Expression vectors include one or more expressin control sequences, such as a promoter, operably linked to the coding sequence such that the peptide is expressed in suitable host cells that contain the expression vector. In one embodiment, the peptide-related polynucleotide is encoded in a plasmid or vector, which may be derived from an adeno-associated virus (AAV). The AAV may be a recombinant AAV virus and may comprise a capsid serotype such as, but not limited to, of AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ, and AAV-D.18. As a non-limiting the capsid of the recombinant AAV virus is AAV2. As a non-limiting example, the capsid of the recombinant AAV vitas is AAVrh10. As a non-limiting example, the capsid of the recombinant AAV virus is AAV9(hu 14). As a non-limiting example, the capsid of the recombinant AAV virus is AAV-DJ. As a non-limiting example, the capsid of the :recombinant AAV virus is AAV9/47. As a non-limiting example, the capsid of the recombinant AAV virus is AAV-DJ8. An embodiment comprises the nucleotide equivalents of the peptide sequences of SEQ ID NOs: 1-38.

A person skilled in the art may recognize that a target cell may require a specific promoter including but not limited to a promoter that is species specific, inducible, tissue- specific, or cell cycle-specific Pan et al, Nat. Med. 3:1145-9 (1997); the contents of which are herein incorporated by reference in its entirety).

As used herein, a “vector” is any molecule or moiety which transports, transduces or otherwise acts as a carrier of a heterologous molecule such as the polynucleotides of the invention. A “viral vector” is a vector which comprises one or more polynucleotide regions encoding or comprising payload molecule of interest, e.g., a transgene, a polynucleotide encoding a polypeptide or multi-polypeptide. Viral vectors of the present invention may be produced recombinantly and may be based on adeno--associated virus (AAV) parent or reference sequence. Serotypes which may be useful in the present invention include any of those arising from AAV1, AAV 2, AAV3, AAV4, AAV5, AAV 6, AAV7, AAV8, AAV9, AAV9.47, AAV9(hul4), AAV10, AAV11, AAV 12, AAVrh8, AAVrh10, AAV-DJ, and AAV-D,18.

In one embodiment, the serotype which may be useful in the present invention may be AAV-DM. The amino acid sequence of AAV-DJ8 may comprise two or more mutations in order to remove the heparin binding domain (HBD). As a non-limiting example, the AAV-DJ sequence described as SEQ ID NO:1 in U.S. Pat. No. 7,588,772, the contents of which are herein incorporated h reference in its entirety, may comprise two mutations: (1) R587Q where arginine (R; arg) at amino acid 587 is changed to glutamine (Q; gin) and (2) R590T where arginine (R; arg) at amino acid 590 is changed to threonine (T; thr). As another non-limiting example, may comprise three mutations: (1) K406R where lysine (K; lys) at amino acid 406 is changed to arginine (R; arg), (2) R587Q where arginine (R; arg) at amino acid 587 is changed to glutamine (Q; gin) and (3) R5901 where arginine (R; arg) at amino acid 590 is changed to threonine (T; thr).

AAV vectors may also comprise self-complementary AAV vectors (scAAVs). scAAV vectors contain both DNA strands which anneal together to form double stranded DNA. By skipping second strand synthesis, scAAVs allow for rapid expression in the cell.

In one embodiment, the pharmaceutical composition comprises a recombinant adeno-associated virus (AAV) vector comprising an AAV capsid and an AAV vector genome. The AAV vector genome may comprise at least one peptide related polynucleotide described herein, such as. but not limited to, SEQ ID NOs: 1-38 or variants having at least 95% identity thereto. The recombinant AAV vectors in the pharmaceutical composition may have at least 70% which contain an AAV vector genome.

In one embodiment, the pharmaceutical composition comprises a recombinant adeno-associated virus (AAV) vector comprising an AAV capsid and an AAV vector genome. The AAV vector genome may comprise at least one peptide related polynucleotide described herein, such as, but not limited to, SEQ ID NOs: 1-38 or variants having at least 95% identity thereto, plus an additional N-terminal proline. The recombinant AAV vectors in the pharmaceutical composition may have at least 70% which contain an AAV vector genome.

In one embodiment, the viral vector comprising a peptide-related polynucleotide may be administered or delivered using the methods for the delivery of AAV virions described in European Patent Application No. EP1857552, the contents of which are herein incorporated by reference in its entirety.

In one embodiment, the viral vector comprising, a peptide-related polynucleotide may be administered or delivered using the methods for delivering proteins using AAV vectors described in European Patent Application No. EP2678433, the contents of which are herein incorporated by reference in its entirety.

In one embodiment, the viral vector comprising a peptide-related polynucleotide may be administered or delivered using the methods for delivering DNA molecules using AAV vectors described in U.S. Pat. No. 5,858,351, the contents of which are herein incorporated by reference in its entirety.

In one embodiment, the viral vector comprising a peptide-related polynucleotide may be administered or delivered using the methods for delivering DNA to the bloodstream described in U.S. Pat. No. 6,211,163, the contents of which are herein incorporated by reference in its entirety.

In one embodiment, the viral vector comprising a peptide-related polynucleotide may be administered or delivered using the methods for delivering AAV virions described in U.S. Pat. No. 6,325,998, the contents of which are herein incorporated by reference in its entirety.

In one embodiment, the viral vector comprising a peptide-related polynucleotide may be administered or delivered using the methods for delivering a payload to the central nervous system described in U.S. Pat. No. 7,588,757, the contents of which are herein incorporated by reference in its entirety.

In one embodiment, the viral vector comprising a peptide-related polynucleotide may be administered or delivered using the methods for delivering a payload described in U.S. Pat. No. 8,283,151, the contents of which are herein incorporated by reference in its entirety.

In one embodiment, the viral vector comprising a peptide-related polynucleotide may be administered or delivered using the methods for delivering a payload using a glutamic acid decarboxylase (GAD) delivery vector described in International Patent Publication No. WO2001089583, the contents of which are herein incorporated by reference in its entirety,

In one embodiment, the viral vector comprising a peptide-related polynucleotide may be administered or delivered using the methods for delivering a payload to neural cells described in International Patent Publication No. WO2012057363, the contents of which are herein incorporated by reference in its entirety.

In one embodiment, the viral vector comprising a peptide-related poly-nucleotide may be administered or delivered using the methods for delivering a payload to cells described in U.S. Pat. No. 9,585,971, the contents of which are herein incorporated by reference in its entirety.

In one embodiment, the viral vector comprising a peptide-related polynucleotide may be administered or delivered using the methods for delivering a payload to cells described in Deverman et al. Nature Biotechnology, 34, 204-09 (2016).

In one embodiment, the viral vector comprising a peptide-related polynucleotide may be administered or delivered using the methods for the delivery of AAV virions described in U.S. Pat. Nos. 7,198,951 (adeno-assoicated virus (AAV) serotype 9 sequences, vectors containing same, and uses therefor), U.S. Pat. No. 9,217,155 (isolation of novel AAV's and uses thereof), WO2011126808 (pharmacologically induced transgene ablation system), U.S. Pat. No. 6,015,709 (transcriptional activators, and compositions and uses related thereto). U.S. Pat. No. 7,094,604 (Production of pseudotyped recombinant AAV virions), International Patent Publication No, WO 2016/126993 (anti-tau constructs), U.S. Pat. No. 7,094,604 (recombinant AAV capsid protein), U.S. Pat. No. 8,292,769 (Avian adenoasssocited vim (aaav) and uses thereof), U.S. Pat. No. 9,102,949 (CNS targeting aav vectors andmethods of use thereof), US Patent Publication No. 2016/0120960 (adeno-associated virus mediated gene transfer to the central nervous system), international Patent Publication No. WO 2016/073693 (A ADC polynucleotides for the treatment of parkinson's disease), International Patent Publication No. WO 2015/168666 (AAV VECTORS FOR RETINAL AND CNS GENE Therapy), US Patent Publication No. 20090117156 (Gene Therapy for Niemann-Pick Disease type A) or International Patent Publication No. WO2005120581 (gene therapy for neurometabolic disorders), all incorporated herein by reference.

The pharmaceutical compositions of viral vectors described herein may be characterized by one or more of bioavailability, therapeutic window and/or volume of distribution.

In some embodiments, peptide-related nucleotides andior peptide-related nucleotide compositions of the present invention may be combined with, coated onto or embedded in a device. Devices may include, but are not limited to stents, pumps, and/or other implantable therapeutic device. Additionally, peptide-related nucleotides and/or peptide--related nucleotide compositions may be delivered to a subject while the subject is using a compression device such as, but not limited to, a compression device to reduce the chances of deep vein thrombosis (DVT) in a subject. The present invention provides for devices which may incorporate viral vectors that encode one or more peptide-related polynucleotide payload molecules. These devices contain in a stable formulation the viral vectors which may be immediately delivered to a subject in need thereof, such as a human patient.

Devices for administration may be employed to deliver the viral vectors comprising an peptide-related nucleotides of the present invention according to single, multi- or split-dosing regimens taught herein.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variation.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein the term “prophylaxis” means prevention of disease or other undesirable/adverse health event or process. The term “prevent” as well as words stemming therefrom, as used herein, does not imply 100% or complete prevention or permanent prevention. Varying degrees of prevention, including delay of onset and/or reduced occurrence (measurable at a population level) are both recognized as benefit or therapeutic effect and scored as prevention. In this respect, the methods described herein can provide any amount of any level of prevention in a subject. Furthermore, the prevention can include prevention (including delay of onset) of one or more conditions or symptoms of the disease. In exemplary aspects, the methods prevent the onset or recurrence by 1 day, 2 days, 4 days, 6 days, 8 days, 10 days, 15 days, 30 days, two months, 4 months, 6 months, 1 year, 2 years, 4 years, or more.

Improvement, preservation, prophylaxis, inhibition-of-deterioration, and prevention are sometimes demonstrable on an individual basis by measuring an indicator, marker, or parameter in question over a minimum clinically meaningful amount of time, which will vary depending on the health assessment in question. Exemplary periods of time include, e.g., 1, 2, 3, 4, 5, 6, 7, 8, 9, 10, 11, 12, 15, 18, 24, 30, 36, 42, 48, 60 or more months, or 1, 2, 3, 4, 5, 6, 7, 8, 9, 10, or more years. Additionally or alternatively, improvement, preservation, prophylaxis, inhibition-of deterioration, and prevention are demonstrable in a population by measuring the parameter in question in the population over time. At the population level, improvement, preservation, prophylaxis, inhibition-of-deterioration, and prevention can be demonstrated statistically, by comparing measurements of a treated population over time with measurements of a control population that did not receive the treatment. While it may not be possible to prove an effect at the individual level for every type of health assessment, such effects often can be demonstrated on a population level through statistical analysis. A dose that is “effective to” improve, preserve, provide prophylaxis, inhibit-deterioration, or prevent can be estimated or demonstrated with a population study. At least for parameters that are difficult or hard to prove at the individual level, an individual who receives the effective dose, over the period required to demonstrate the effect at the population level, is scored as an individual in whom improvement, preservation, prophylaxis, or inhibition-of-deterioration of the healthspan parameter has been achieved.

### Compositions

The pharmaceutical compositions are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or infusions; or kidney dialytic infusion techniques.

In various embodiments, the peptide is admixed with a pharmaceutically acceptable excipients to form a pharmaceutical composition that can be systemically administered to the subject orally or via intravenous injection, intramuscular injection, subcutaneous injection, intraperitoneal injection, transdermal injection, intra-arterial injection, intrasternal injection, intrathecal injection, intraventricular injection, intraurethral injection, intracranial injection, intrasynovial injection or via infusions. The pharmaceutical composition preferably contains at least one component that is not found in nature.

Formulations of a pharmaceutical composition suitable for parenteral administration typically generally comprise the active ingredient combined with a pharmaceutically acceptable excipient, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain carriers such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The present disclosure includes compositions and methods for transdermal or topical delivery, to act locally at the point of application, or to act systemically once entering the body's blood circulation. In these systems, delivery may be achieved by techniques such as direct topical application of a substance or drug in the form of an ointment or the like, or by adhesion of a patch with a reservoir or the like that holds the drug (or other substance) and releases it to the skin in a time-controlled fashion. For topical administration, the compositions can be in the form of emulsions, lotions, gels, creams, jellies, solutions, suspensions, ointments, and transdermal patches. Some topical delivery compositions may contain polyenylphosphatidylcholine (herein abbreviated “PPC”). In some cases, PPC can be used to enhance epidermal penetration. The term “polyenylphosphatidylcholine,” as used herein, means any phosphatidylcholine bearing two fatty acid moieties, wherein at least one of the two fatty acids is an unsaturated fatty acid with at least two double bonds in its structure, such as linoleic acid. Such topical formulations may comprise one or more emulsifiers, one or more surfactants, one or more polyglycois, one or more lecithins, one or more fatty acid esters, or one or more transdermal penetration enhancers. Preparations can include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, organic esters such as ethyl oleate, or fixed oils including synthetic mono or di-glycerides. Aqueous solvents include water, alcoholic/aqueous solutions, emulsions or suspensions, including saline and buffered media. Parenteral vehicles include sodium chloride solution, 1,3-butantioi, Ringer's dextrose, dextrose and sodium chloride, lactated Ringer's or fixed oils, Intravenous vehicles include fluid nd nutrient replenishers, electrolyte replenishers (such as those based on Ringer's dextrose), and the like. Preservatives and other additives may also be present such as, for example, antimicrobials, antioxidants, chelating agents and inert gases and the like.

For example, in one aspect, sterile injectable solutions can be prepared by incorporating a peptide in the required amount in an appropriate solvent with one or a combination of ingredients enumerated above, as required, followed by filtered sterilization. Generally, dispersions are prepared by incorporating the active peptide into a sterile vehicle that contains a basic dispersion medium and the required other ingredients from those enumerated above. In the case of sterile powders for the preparation of sterile injectable solutions, methods of preparation such as vacuum drying and freeze-drying yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. In various embodiments, the injectable compositions will be administered using commercially available disposable injectable devices.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind known in the art. Injectable formulations are in accordance with the disclosure. The requirements for effective pharmaceutical excipients for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the peptides of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the peptides of the disclosure can be formulated as inclusion complexes, such as cyclodextrin inclusion complexes, or liposomes.

The peptide can be administered intranasally or by inhalation, typically in the form of a dry powder (either alone, as a mixture, or as a mixed component particle, for example, mixed with a suitable pharmaceutically acceptable carrier) from a dry powder inhaler, as an aerosol spray from a pressurized container, pump, spray, atomiser (preferably an atomiser using electrohydrodynamics to produce a fine mist), or nebulizer, with or without the use of a suitable propellant, or as nasal drops. The pressurized container, pump, spray, atomizer, or nebulizer generally contains a solution or suspension of a peptide comprising, for example, a suitable agent for dispersing, solubilizing, or extending release of the active, a propellant(s) as solvent. Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules, blisters and cartridges for use in an inhaler or insufflator may be formulated to contain a powder mix of the peptide, a suitable powder base and a performance modifier. Suitable flavors, such as menthol and levomenthol, or sweeteners, such as saccharin or saccharin sodium, may be added to those formulations intended for inhaled/intranasal administration. Formulations for inhaled/intranasal administration may be formulated to be immediate and/or modified release. Modified release formulations include delayed-, sustained-, pulsed-, controlled-, targeted and programmed release. In the case of dry powder inhalers and aerosols, the dosage unit is determined by means of a valve which delivers a metered amount. Units are typically arranged to administer a metered dose or “puff” of a peptide. The overall daily dose will typically be administered in a single dose or, more usually, as divided doses throughout the day.

### Indications

According to one aspect, the peptides are for use in medicine, particularly human medicine. Peptides of the invention can be used for the treatment of fibrosis, For example the peptides may be used for the treatment of lung fibrosis such as idiopathic pulmonary fibrosis. Fibrosis is characterized by the development of excess fibrous connective tissue due at least in part to reparative or reactive processes, such as in response to an injury. In fibrosis, the abnormal accumulation of extracellular matrix proteins can result in scarring and thickening of the affected tissue. Fibrosis can occur in various organs including ale lung, liver, heart, kidney, pancreas, skin, and brain. Various conditions and disorders are accompanied by fibrosis, such as cardiomyoptithies, hypertension, arterial stiffness, chronic hepatitis C infection, Crohn's disease, adult respiratory distress syndrome, and sarcoidosis. Exemplary fibrotic diseases include, but are not limited to, multi-systemic (e.g., systemic sclerosis, multifocal fibrosclerosis, sclerodermatous graft-versus-host disease in bone marrow transplant recipients, nephrogenic systemic fibrosis, or scleroderma) and organ-specific disorders (e.g., fibrosis of the lung, heart, kidney, pancreas, skin, brain, eye and other organs). For example, the fibrosis of the lung can be associated with (e.g., secondary to) one or more of: a disease process, such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder (e.g., sarcoidosis); or an infectious disease (e.g., infection, particularly chronic infection), cystic fibrosis, other diffuse parenchymal lung diseases of different etiologies including iatrogenic drug-induced fibrosis, occupational andlor environmental induced fibrosis, granulomatous diseases (hypersensitivity pneumonia), collagen vascular disease, alveolar proteinosis, langerhans cell granulomatosis, lymphangioleiomyomatosis, inherited diseases (Hermansky-Pudlak Syndrome, neurofibromatosis, metabolic storage disorders, familial interstitial lung disease), Neomycin induced pulmonary fibrosis, asbestos induced pulmonary fibrosis, tubulointerstitium fibrosis, glomerular nephritis, focal segmental glomerular sclerosis, IgA nephropathy, Alport, gut fibrosis, cirrhosis, alcohol induced liver fibrosis, toxic/drug, induced liver fibrosis, hemochromatosis, nonalcoholic steatohepatitis (NASH), biliary duct injury, primary biliary cirrhosis, infection induced liver fibrosis, viral induced liver fibrosis, and autoimmune hepatitis, corneal scarring, hypertrophic scarring, Dupuytren disease, keloids, cutaneous fibrosis, cutaneous scleroderma, spinal cord injury/fibrosis, myelofibrosis, vascular restenosis, atherosclerosis, arteriosclerosis, Peyronie's disease, or chronic lymphocytic thyroiditis fibrosis.

In one embodiment, the fibrotic condition of the lung is associated with an autoimmune connective tissue disorder (e.g., scleroderma or lupus, e.g., SLE).

In other embodiments, pulmonary fibrosis includes, but is not limited to, pulmonary fibrosis associated with chronic obstructive pulmonary disease (COPD), acute respiratory distress syndrome (ARDS), scleroderma, pleural fibrosis, chronic asthma, acute lung syndrome, amyloidosis, bronchopulmonary dysplasia, Caplan's disease, Dressler's syndrome, histiocytosis X, idiopathic pulmonary haemosiderosis, lymphangiomyomatosis, mitral valve stenosis, polymyositis, pulmonary edema, pulmonary hypertension (e.g., idiopathic pulmonary hypertension (IPH)), pneumoconiosis, radiotherapy (e.g., radiation induced fibrosis), rheumatoid disease, Shaver's disease, systemic lupus erythematosus, systemic sclerosis, tropical pulmonary eosinophilia, tuberous sclerosis, Weber-Christian disease, Wegener's granulomatosis, Whipple's disease, or exposure to toxins or irritants (e.g., pharmaceutical drugs, such as amiodarone, Neomycin, busulphan, carmustine, chloramphenicol, hexamethonium, methotrexate, methysergide, mitomycin C , nitrofurantoin, penicillamine, peplomycin, or practolol; or inhalation of talc or dust, e.g., coal dust, silica). In certain embodiments, the pulmonary fibrosis is associated with an inflammatory disorder of the lung, e.g. one or both of asthma or COPD.

A “fibrosis-associated condition” means any condition that is related to fibrosis. Thus, fibrosis-associated conditions may be caused by, he concomitant with, or cause fibrosis. Chronic kidney disease is an example of a fibrosis-associated condition.

According to another embodiment, the peptides are coadministered or co-formulated with other known chemotherapeutic agents and/or anti-inflammatory agents.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

In one embodiment, antifibrotic agents are co-administered, administered sequentially, or co-prescribed (such that medicines are requested by a prescribing physician to he taken in some sequence as combination therapy to treat the same disease).

It is to be noted that dosage values may vary with the type and severity of the condition to be ameliorated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular peptide employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

The dose of the peptide of the present disclosure also will be determined by the existence, nature and extent of any adverse side effects that might accompany the administration of a particular peptide of the present disclosure. Typically, the attending physician will decide the dosage of the peptide of the present disclosure with which to treat each individual patient, taking into consideration a variety of factors, such as age, body weight, general health, diet, sex, peptide of the present disclosure to he administered, route of administration, and the severity of the condition being treated. By way of example and not intending to be limiting, the dose of the peptide of the present disclosure can be about 0.0001 to about 100 mg/kg body weight of the subject being treated/day, from about to about 10 mg/kg body weight/day, or about 0.01 mg to about 1 mg/kg body weight/day. The peptide can be administered in one or more doses, such as from 1 to 3 doses.

In some embodiments, the pharmaceutical composition comprises any of the analogs disclosed herein at a purity level suitable for administration to a patient. In some embodiments, the analog has a purity level of at least about 90%, preferably above about 95%, more preferably above about 99%, and a pharmaceutically acceptable diluent, carrier or excipient.

The pharmaceutical compositions may be formulated to achieve a physiologically compatible pH. In some embodiments, the pH of the pharmaceutical composition may be at least 5, or at least 6, or at least 7, depending on the formulation and route of administration.

In various embodiments, single or multiple administrations of the pharmaceutical compositions are administered depending on the dosage and frequency as required and tolerated by the subject. In any event, the composition should provide a sufficient quantity of at least one of the peptide disclosed herein to effectively treat the subject. The dosage can be administered once but may be applied periodically until either a therapeutic result is achieved or until side effects warrant discontinuation of therapy.

The dosing frequency of the administration of the peptide pharmaceutical composition depends on the nature of the therapy and the particular disease being treated. The administration may be once, twice, three times or four times daily, for the peptide. Treatment of a subject with a therapeutically effective amount of a peptide, can include a single treatment or, preferably, can include a series of treatments. In a preferred example, a subject is treated with peptide daily, one time per week or biweekly.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and. equivalents as may be included within the spirit and scope of the embodiments of the present disclosure as defined by the appended claims.

### EMBODIMENTS

The embodiments listed below are presented in numbered. form for convenience and for ease and clarity of reference in referring back to multiple embodiments. The embodiments include:
1. A method for treating fibrosis in a patient in need of such treatment, comprising administering to the patient a pharmacologically effective amount of a first antifibrotic agent and a peptide antifibrotic agent, said peptide fibrotic agent comprising an amino acid sequence of Formula I:

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK, -LAGK; or -L(dA); and X¹⁴ is N or L; or C-terminal acids or amides, or N-acetyl derivatives thereof; or pharmaceutically acceptable salts thereof.
2. The method of Embodiment I wherein the fibrosis is any of cirrhosis of the liver; pulmonary fibrosis, idiopathic pulmonary fibrosis; fibrosis following myocardial infarction; CNS fibrosis following a stroke, neurodegenerative disorders, Alzheimer's Disease, multiple sclerosis; proliferative vitreoretinopathy (PVR), arthritis; adhesions, nephrogenic systemic fibrosis; myocardial fibrosis; hepatic fibrosis; epidural fibrosis (failed back surgery syndrome); endomyocardial fibrosis; tubulointerstitial fibrosis; renal interstitial fibrosis; mediastinal fibrosis; retroperitoneal fibrosis; penile fibrosis; oral submucous; kidney fibrosis; idiopathic pulmonary upper lobe fibrosis; congenital hepatic fibrosis; postlaminotorny fibrosis; painful disc fibrosis; graft fibrosis; atrial fibrosis; corneal subepithelial fibrosis; congenital orbital fibrosis; bone fibrosis; peritoneal fibrosis; nephrogenic systemic fibrosis; non-cirrhotic portal fibrosis; pulmonary tuberculosis, disease-related pulmonary apical fibrosis in ankylosing spondylitis; colorectal fibrosis; periglomerular fibrosis/atubular glomeruli; basal fibrosis syndrome; tissue fibrosis; and massive neck fibrosis.
3. The method of Embodiment 1 wherein the fibrosis is idiopathic pulmonary fibrosis.
4. The method of Embodiment 1 wherein the fibrosis is scleroderma or systemic sclerosis.
5. The method of Embodiment 1 wherein the peptide antifibrotic agent comprises an amino acid sequence selected from MR VIRMCLGVGLLGDLAG (SEQ ID NO: 2); RVIRMCLGVGLLGDLAG (SEQ ID NO: 3); RVIRMCLGVGLLGDL(dA)G (SEQ ID NO: 4); RVIRMCLNVGLLGEL(dA)G (SEQ ID NO: 5); RVIR(Nle)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNI.GEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIR CLGVGLI-GDLAG (SEQ ID NO: 16); RVIRMCLGVGLLGDLAGK{PEG12} (SEQ ID NO: 17); {PEG12}KRVIRMCLAVGLLGEL(dA)E (SEQ ID NO: 18); RVIRMCLNVGLEGEL(dA) (SEQ NO: 19); RVIRMCLNVGLNGEL(dA)E (SEQ ID NO: 20); RVIRMCLNVGLNGE (SEQ ID NO: 21); RVIRMCLNNGLNGEL(dA)}G (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK{PEG12} (SEQ ID NO: 25); RVIRACLGVGLLGDL(dA)GK{PEG12} (SEQ ID NO: 29); RVIR(Ne)CLGVGLI-GDL(dA)GK (SEQ ID NO: 33); RVIR(Nle)CLGVGLLGDL(dA)GK({PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLI,GDLAGMPECH21 (SEQ ID NO: 38); or pharmaceutically acceptable salts thereof.
6. The method of any one of Embodiments 1-5, wherein the peptide antifibrotic agent comprises a peptide dirtier comprised of peptides comprising an amino acid sequence of Formula I.
7. The method of any one of Embodiments 1-6, wherein the peptide antifibrotic agent comprises a peptide or dimer thereof that is is derivatized via acetylation, pegylation, biotinylation or acylation.
8. The method of any one of Embodiments 1-7, wherein the peptide antifibrotic agent is selected from

```
(SEQ ID NO: 26)
RVIRMCLGVGLLGDLAG
          |
RVIRMCLGVGLLGDLAG;
(SEQ ID NO: 27)
RVIRMCLNVGLLGEL(dA)G
          |
RVIRMCLNVGLLGEL(dA)G;
(SEQ ID NO: 28)
RVIRMCLGVGLLGDLAGK{PEG12}
          |
RVIRMCLGVGLLGDLAGK{PEG12};
(SEQ ID NO: 30)
RVIRACLGVGLLGDL(dA)GK{PEG12}
          |
RVIRACLGVGLLGDL(dA)GK{PEG12};
(SEQ ID NO: 32)
RVIR(Nle)CLGVGLLGDL(dA)GK{PEG12}
          |
RVIR(Nle)CLGVGLLGDL(dA)GK{PEG12};
(SEQ ID NO: 33)
RVIR(Nle)CLGVGLLGDL(dA)GK;
(SEQ ID NO: 34)
RVIR(Nle)CLGVGLLGDL(dA)GK{PEG12};
(SEQ ID NO: 35)
RVIRACLGVGLLGDL(dA)GK;
(SEQ ID NO: 36)
RVIRACLGVGLLGDLAGK{PEG12}
          |
RVIRACLGVGLLGDLAGK{PEG12};
(SEQ ID NO: 37)
RVIRACLGVGLLGDLAGK;
or
(SEQ ID NO: 38)
RVIRACLGVGLLGDLAGK{PEG12}.
```

9. The method of any one of Embodiments 1-8 wherein the peptide antifibrotic agent is

```
(SEQ ID NO: 30)
RVIRACLGVGLLGDL(dA)GK{PEG12}
          |
RVIRACLGVGLLGDL(dA)GK{PEG12};
or
(SEQ ID NO: 32)
RVIR(Nle)CLGVGLLGDL(dA)GK{PEG12}
                  |
RVIR(Nle)CLGVGLLGDL(dA)GK{PEG12}.
```

10. The method of any one of Embodiments 1-9, wherein the first antifibrotic agent comprises one or more agents selected from anti-LOXL2 antibodies, recombinant pentraxins, PDGF inhibitors, PDGF receptor inhibitors, FOF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-6, αVβ1/αVβ6 inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, Jun N-terminal kinase (JNK) 1/2 inhibitors, JNK1 inhibitors, NOX 1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.
11. The method Embodiment 10 wherein the first antifibrotic agent comprises one or mare agents selected from nintedanib, pirfenidone, BBT-877 (Bridge Biotherapeutics and Boehringer Ingelheim), GLPG1690 (Galapagos), BMS-986,020 (Bristol Myers Squibb), BMS-986,278 (Bristol Myers Squibb), Painreviumab (FibroGen), N-acetyl cysteine (NAC), PRM-151 (Promedior), lanaturnab (Novartis), BLD-2660 (Blade), PLN-74809 (Pliant), ND-L02-s0201 (Nitto Denko), CC-90001 (Celgene), tipelukast (MedicNova, MN-001), Setanaxib (Genkyotex, GKT137831), KD025 (Kadmon), and GB0139 (Galecto).
12. The method of Embodiment wherein the first antifibrotic agent comprises one or more agents selected from pirfenidone and nintedanib.
13. The method of any one of Embodiments 1-12, wherein the first antifibrotic agent and the peptide antifibrotic, agent are co-administered in the same formulation.
14. The method of any one of Embodiments 1-12, wherein the first antifibrotic agent and the peptide antifibrotic agent are administered sequentially.
15. A combination of a first antifibrotic agent and an antifibrotic peptide comprising an amino acid sequence of formula I:

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK, -LAGK; or -L(dA); and X¹⁴ is N or L; or C-terminal acids or amides, or N-acetyl derivatives thereof; or pharmaceutically acceptable salts thereof; or N-acetyl derivatives thereof; or pharmaceutically acceptable salts thereof.
16. The combination of Embodiments 15, wherein the first antifibrotic agent and the antifibrotic peptide are co-formulated in a composition.
17. The combination of Embodiment 15, wherein the first antifribrotic agent and the antifibrotic peptide are packaged together in a kit.
18. The combination according to Embodiments 17, wherein the first antifibrotic agent and the antifibrotic, peptide are packaged in unit dosage form in the kit.
19. The combination of any one of Embodiments 15-18, wherein the antifibrotic peptide comprises an amino acid sequence selected from MRVIRMCLGVGLLGDLAG (SEQ ID NO: 2); RVIRMCLGVGLLGDLAG (SEQ ID NO: 3); RVIRMCLGVGLLGDL(dA)G (SEQ ID NO: 4); RVIRMCLNVGLLGEL(dA)G (SEQ ID NO: 5); RVIR(N1e)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNI.GEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIRMCLGVGLLGDLAG (SEQ ID NO: 16); RVIRMCLGVGLLGDLAGK{PEG12} (SEQ ID NO: 17); {PEG12}KRV1RMCLNVGLLGEL(dA)E (SEQ ID NO: 18); RVIRMCLNVGLEGEL(dA) (SEQ ID NO: 19); RVIRMCLNVGLNGEL(dA)E (SEQ ID NO: 20); RVIRMCLNVGLNGE (SEQ ID NO: 21); RVIRNICLNNGLNGEL(dA))G (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK{PEG12} (SEQ ID NO: 25); RVIRACLGVGLLGDL(dA)GK{PEG12} (SEQ ID NO: 29); RVIR(Nle)CLGVGLLGDL(dA)GK (SEQ ID NO: 33); RVIR(Nle)CLGVGLLGDI-(dA)GK{PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLLGDLAGK{PEG12} (SEQ ID NO: 38); or pharmaceutically acceptable salts thereof.
20. The combination of any one of Embodiments 15-18, wherein the antifibrotic peptide is selected from

```
(SEQ ID NO: 26)
RVIRMCLGVGLLGDLAG
          |
RVIRMCLGVGLLGDLAG;
or
(SEQ ID NO: 27)
RVIRMCLNVGLLGEL(dA)G
          |
RVIRMCLNVGLLGEL(dA)G;
or
(SEQ ID NO: 28)
RVIRMCLGVGLLGDLAGK{PEG12}
          |
RVIRMCLGVGLLGDLAGK{PEG12};
(SEQ ID NO: 30)
RVIRACLGVGLLGDL(dA)GK{PEG12}
          |
RVIRACLGVGLLGDL(dA)GK{PEG12};
and
(SEQ ID NO: 32)
RVIR(Nle)CLGVGLLGDL(dA)GK{PEG12}
                  |
RVIR(Nle)CLGVGLLGDL(dA)GK{PEG12}.
```

21. The combination of any one of Embodiments 15-20, wherein the first antifibrotic agent comprises one or more agents selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGE inhibitors, TGF-β inhibitors, anti-BAFF-R antibodies, calpain inhibitors, antibodies targeting integrin alpha-v beta-6, αVβ1/αVβ6 inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, Jun N-terminal kinase (INK) 1/2 inhibitors, JNK1 inhibitors, NOX1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.
22. The combination of any one of Embodiments 15-20, wherein the first antifibrotic agent comprises one or more agents selected from nintedanib, pirfenicone, BBT-877 (Bridge Biotherapeutics and Boehringer Ingelheim), GLPG1690 (Galapagos), BMS-986,020 (Bristol Myers Squibb), BMS-986,278 (Bristol Myers Squibb), Parnreviurnab (FibroGen), N-acetyl cysteine (NAC), PRM-151 (Promedior), lanaiumab (Novartis), BLD-2660 (Blade), PLN-74809 (Pliant), ND-L02-s0201 (Nitto Denko), CC-90001(Celgene), tipelukast (MedicNova, MN-001), Setanaxib (Genkyotex, GK1137831), KD025 (Kadmon), and GB0139 (Galecto).
23. The combination of any one of Embodiments 15-20, wherein the first antifibrotic agent is one or more agents selected from pirfenidone or nintedanib.
24. A peptide of Formula I comprising

```
(SEQ ID NO: 32)
RVIR(Nle)CLGVGLLGDL(dA)GK{PEG12}
                  |
RVIR(Nle)CLGVGLLGDL(dA)GK{PEG12};
(SEQ ID NO: 33)
RVIR(Nle)CLGVGLLGDL(dA)GK;
(SEQ ID NO: 34)
RVIR(Nle)CLGVGLLGDL(dA)GK{PEG12};
(SEQ ID NO: 35)
RVIRACLGVGLLGDL(dA)GK;
(SEQ ID NO: 36)
RVIRACLGVGLLGDLAGK{PEG12}
          |
RVIRACLGVGLLGDLAGK{PEG12};
(SEQ ID NO: 37):
RVIRACLGVGLLGDLAGK
or
(SEQ ID NO: 38)
RVIRACLGVGLLGDLAGK{PEG12}.
```

25. Use of the combination according to any one of Embodiments 15-23, in the manufacture of a medicament for treating fibrosis.
26. The combination according to any one of Embodiments 15-23 for use in treating fibrosis.
27. A medicament for treating fibrosis in a patient in need of such treatment, comprising administering to the patient a pharmacologically effect amount of a composition according to any one of Embodiments 15-23.

The embodiments further include the following:
201. A method for treating fibrosis in a patient in need of such treatment, the method comprising administering to the patient a phamacologically effective amount of a first antifibrotic agent and a peptide antibrotic agent, or analog of said peptide, or derivative thereof, or pharmaceutically acceptable salt thereof,
   said peptide antifibrotic agent comprising an amino acid sequence of Formula I:

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK, -LAGK; or -L(dA); and X¹⁴ is N or L; or C-terminal acids or amides, or N-acetyl derivatives thereof; or pharmaceutically acceptable salts thereof.
202. A combination comprising a first antifibrotic agent and a peptide antibrotic agent, or analog of said peptide, or derivative thereof, or pharmaceutically acceptable salt thereof, for use in the treatment of fibrosis in a patient in need of such treatment,
   said peptide antifibrotic agent comprising an amino acid sequence of Formula I:

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK, -LAGK; or -L(dA); and X¹⁴ is N or L; or C-terminal acids or amides, or N-acetyl derivatives thereof; or pharmaceutically acceptable salts thereof.
203. The method of embodiment 201 or the combination of embodiment 202, wherein the fibrosis is any of cirrhosis of the liver; pulmonary fibrosis, idiopathic pulmonary fibrosis; fibrosis following myocardial infarction; CNS fibrosis following a stroke, neurodegenerative disorders, Alzheimer's Disease, multiple sclerosis; proliferative vitreoretinopathy (PVR), arthritis; adhesions, nephrogenic systemic fibrosis; myocardial fibrosis; hepatic fibrosis; epidural fibrosis (failed back surgery syndrome); endomyocardial fibrosis; tubulointerstitial fibrosis; renal interstitial fibrosis; mediastinal fibrosis; retroperitoneal fibrosis; penile fibrosis; oral submucous; kidney fibrosis; idiopathic pulmonary upper lobe fibrosis; congenital hepatic fibrosis; postlaminotomy fibrosis; painful disc fibrosis; graft fibrosis; atrial fibrosis; corneal stibepitlielial fibrosis; congenital orbital fibrosis; bone fibrosis; peritoneal fibrosis; nephrogenic systemic fibrosis; non-cirrhotic portal fibrosis; pulmonary tuberculosis, disease-related pulmonary apical fibrosis in ankylosing spondylitis; colorectal fibrosis; periglomerular fibrosislatubuiar glomeruli; basal fibrosis syndrome; tissue fibrosis; and massive neck fibrosis.
204. The method of embodiment 201 or the combination of embodiment 202, wherein the fibrosis is idiopathic pulmonary fibrosis.
205. The method of embodiment 201 or the combination of embodiment 202, wherein the fibrosis is scleroderma or systemic sclerosis.
206. The method of embodiment 201 or the combination of embodiment 202, wherein the peptide antifibrotic agent comprises an amino acid sequence selected from MRVIRMCLGVGLLGDLAG (SEQ ID NO: 2); RVIRMCLGVGLLGDLAG (SEQ ID NO: 3); RVIRMCLGVGLLGDL(dA)G (SEQ ID NO: 4); RVIRMCLNVGLLGEL(dA)G (SEQ ID NO: 5); RVIR(Nle)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNLGEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIRMCLGVGLLGDLAG (SEQ ID NO: 16); RVIRMCLGVGLLGDLAGK{PEG12} (SEQ ID NO: 17); {PEG12}KRVIRMCLNVGLLGEL(dA)E (SEQ ID NO: 18); RVIRMCLNVGLEGEL(dA) (SEQ ID NO: 19); RVIRMCLNVGLNGEL(dA)E (SEQ ID NO: 20); RVIRMCLNVGLNGE (SEQ ID NO: 21); RVIRMCLNNGLNGEL(dA)IG (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK {PEG12} (SEQ ID NO: 25); RVIRACLGVGLLGDL(dA)GK{PEG12} (SEQ ID NO: 29); RVIR(Nle)CLGVGLLGDL(dA)GK (SEQ ID NO: 33); RVIR(Nle)CLGVGLLGDL(dA)GK{PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLLGDLAGK{PEG12} (SEQ ID NO: 38); or pharmaceutically acceptable salts thereof.
207. The method of any one of embodiments 201 and 203-206, or the combination of any one of embodiments 202-206, wherein the peptide antifibrotic agent comprises a peptide dimer comprised of peptides comprising an amino acid sequence of Formula I.
208. The method of any one of embodiments 201 and 203-207, or the combination of any one of embodiments 202-207, wherein the peptide antifibrotic agent comprises a peptide or dimer thereof that is is derivatized via acetylation, pegylation, biotinylation or acylation.
209. The method of any one of embodiments 201 and 203-208, or the combination of any one of embodiments 202-208, wherein the peptide antifibrotic agent is selected from

```
(SEQ ID NO: 26)
RVIRMCLGVGLLGDLAG
          |
RVIRMCLGVGLLGDLAG;
(SEQ ID NO: 27)
RVIRMCLNVGLLGEL(dA)G
          |
RVIRMCLNVGLLGEL(dA)G;
(SEQ ID NO: 28)
RVIRMCLGVGLLGDLAGK{PEG12}
          |
RVIRMCLGVGLLGDLAGK{PEG12};
(SEQ ID NO: 30)
RVIRACLGVGLLGDL(dA)GK{PEG12}
          |
RVIRACLGVGLLGDL(dA)GK{PEG12};
(SEQ ID NO: 32)
RVIR(Nle)CLGVGLLGDL(dA)GK{PEG12}
                  |
RVIR(Nle)CLGVGLLGDL(dA)GK{PEG12};
(SEQ ID NO: 33)
RVIR(Nle)CLGVGLLGDL(dA)GK;
(SEQ ID NO: 34)
RVIR(Nle)CLGVGLLGDL(dA)GK{PEG12};
(SEQ ID NO: 35)
RVIRACLGVGLLGDL(dA)GK;
(SEQ ID NO: 36)
RVIRACLGVGLLGDLAGK{PEG12}
          |
RVIRACLGVGLLGDLAGK{PEG12};
(SEQ ID NO: 37)
RVIRACLGVGLLGDLAGK;
or
(SEQ ID NO: 38)
RVIRACLGVGLLGDLAGK{PEG12}.
```

210. The method of any one of embodiments 201 and 203-209, or the combination of any one of embodiments 202-209, wherein the peptide antifibrotic agent is

```
(SEQ ID NO: 30)
RVIRACLGVGLLGDL(dA)GK{PEG12}
          |
RVIRACLGVGLLGDL(dA)GK{PEG12};
or
(SEQ ID NO: 32)
RVIR(Nle)CLGVGLLGDL(dA)GK{PEG12}
                  |
RVIR(Nle)CLGVGLLGDL(dA)GK{PEG12}.
```

211. The method of any one of embodiments 201 and 203-210, or the combination of any one of embodiments 202-210, wherein the first antifibrotic agent comprises one or more agents selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-6, αVβ1/αVβ6 inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, IRE-1 inhibitors, P2X3 antagonists, SRC inhibitors, Jun N-terminal kinase (JNK) 1/2 inhibitors, JNK1 inhibitors, NOX1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.
212. The method of any one of embodiments 201 and 203-210, or the combination of any one of embodiments 202-210, wherein the first antifibrotic agent comprises one or more agents selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-6, αVβ1/αVβ6 inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, Jun N-terminal kinase (JNK) 1/2 inhibitors, JNK1 inhibitors, NOX 1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.
213. The method of any one of embodiments 201 and 203-210, or the combination of any one of embodiments 202-210, wherein the first antifibrotic agent comprises a PDGF receptor inhibitor or a TGF-β inhibitor.
214. The method of embodiment 213 or the combination of embodiment 213, wherein the first antifibrotic agent comprises a TGF-β inhibitor selected from vactosertib (TEW7197), pirfenidone, and galunisertib.
215. The method of embodiment 213 or the combination of embodiment 213 wherein the first antifibrotic agent comprises a PDGF receptor inhibitor selected from nintedanib, sunitinib, imatinib, and sorafenib.
216. The method of any one of embodiments 201 and 203-210, or the combination of any one of embodiments 202-210, wherein the first antifibrotic agent comprises one or more agents selected from nintedanib, pirfenidone, indolinone, simtuzumab, (Gilead, GS-6624), IW001 (ImmuneWorks), PRM-151 (recombinant human pentraxin-2 protein, Genetech, Promedior), TANZISERTIB (Celgene, CC-930), imatinib, STX-100 (Biogen), dectrekumab (Novartis, QAX576), pamrevlumab (FibroGen), carlumab (Janssen, CNTO-888), SM-04646 (Samumed), N-acetylcysteine (NAC), CC-90001 (Celgene), BMS-986,020 (Bristol Myers Squibb), BMS-986,278 (Bristol Myers Squibb), BBT-877 (Bridge Biotherapeutics and Boehringer Ingelheim), GLPG1690 (Galapagos), BI 1015550 (Boehringer Ingelheim), Gefapixant, Setogepram (ProMetic, PBI-4050), tipelukast (MediciNova), GB 2064 (GALECTO, PAT-1251), lanalumab (Novartis), BLD-2660 (Blade), PLN-74809 (Pliant), ND-L02-s0201 (Nitto Denko), setanaxib (GKT137831, Genkyotex), belumosudil (KD025, Kadmon), GB0139 (Galecto), BNC₁₀₂₁ (BONAC/Toray, TRK-250), ORIN1001 (Fosun), sildenafil, macitentan, bosentan, lebrikizumab, valganciclovir, letemovir, minocycline, gefapixant, zileuton, NIP292 (CR Pharma), voxelotor (GBT446), HEC_{825,} HEC_{6840,} saracatinab, and CC₉₀₀₀₁ (BMS).
217. The method of any one of embodiments 201 and 203-210, or the combination of any one of embodiments 202-210, wherein the first antifibrotic agent comprises one or more agents selected from nintedanib, pirfenidone, indolinone, simtuzumab, tanzisertib, imatinib, dectrekumab, pamrevlumab, carlumab, N-acetylcysteine (NAC), gefapixant, Setogepram, tipelukast, lanalumab, setanaxib, belumosudil, sildenafil, macitentan, bosentan, lebrikizumab, valganciclovir, letemovir, minocycline, gefapixant, zileuton, voxelotor and saracatinab.
218. The method of any one of embodiments 201 and 203-210, or the combination of any one of embodiments 202-210, wherein the first antifibrotic agent comprises one or more agents selected from

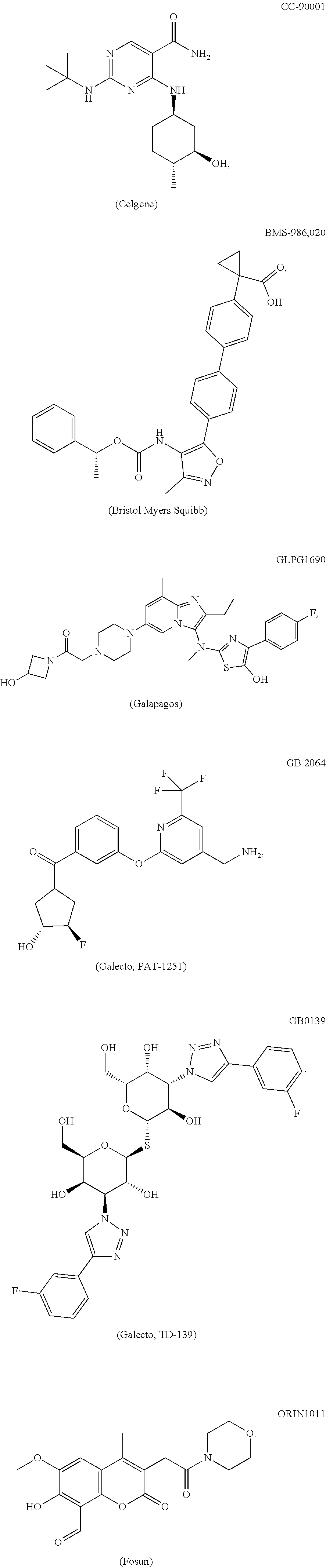 [SMILES: C[C@@H]1CC[C@@H](Nc2nc(NC(C)(C)C)ncc2C(N)=O)C[C@H]1O ; Cc1noc(-c2ccc(-c3ccc(C4(C(=O)O)CC4)cc3)cc2)c1NC(=O)O[C@H](C)c1ccccc1 ; CCc1nc2c(C)cc(N3CCN(CC(=O)N4CC(O)C4)CC3)cn2c1N(C)c1nc(-c2ccc(F)cc2)c(O)s1 ; NCc1cc(Oc2cccc(C(=O)C3C[C@@H](O)[C@H](F)C3)c2)nc(C(F)(F)F)c1 ; OC[C@H]1O[C@@H](S[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)[C@H]1O ; COc1cc2c(C)c(CC(=O)N3CCOCC3)c(=O)oc2c(C=O)c1O]

219. The method of any one of embodiments 201 and 203-210, or the combination of any one of embodiments 202-210, wherein the first antifibrotic agent comprises one or more agents selected from pirfenidone and nintedanib.
220. The method of any one of embodiments 201 and 203-219, or the combination of any one of embodiments 202-219, wherein the first antifibrotic agent and the peptide antifibrotic agent are formulated in a medicament for co-administration.
221. The method of any one of embodiments 201 and 203-219 that comprises administering the first antifibrotic agent and the peptide antifibrotic agent simultaneously.
222. The method of any one of embodiments 201 and 203-219 that comprises administering the first antifibrotic agent and the peptide antifibrotic agent sequentially.
223. The combination of any one of embodiments 202-219, wherein the first antifibrotic agent and the peptide antifibrotic agent are formulated for sequential administration.
224. A combination of a first antifibrotic agent and a peptide antifibrotic agent comprising a peptide comprising an amino acid sequence of formula I:

(SEQ ID NO: 31)
X¹-R-X²-IR-X³-X⁴-L-X⁵-X⁶-G-X¹⁴-X⁷-G-X⁸-X⁹ (I)

wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK, -LAGK; or -L(dA); and X¹⁴ is N or L; or C-terminal acids or amides, or N-acetyl derivatives thereof; or pharmaceutically acceptable salts thereof; or N-acetyl derivatives thereof; or pharmaceutically acceptable salts thereof.
225. The combination of embodiments 224, wherein the first antifibrotic agent and the peptide antifibrotic agent are co-formulated in a composition.
226. The combination of embodiment 224, wherein the first antifribrotic agent and the peptide antifibrotic agent are packaged together in a kit.
227. The combination according to embodiments 226, wherein the first antifibrotic agent and the peptide antifibrotic agent are packaged in unit dosage form in the kit.
228. The combination of any one of Embodiments 224-227, wherein the peptide antifibrotic agent comprises a peptide comprising an amino acid sequence selected from MRVIRMCLGVGLLGDLAG (SEQ ID NO: 2); RVIRMCLGVGLLGDLAG (SEQ ID NO: 3); RVIRMCLGVGLLGDL(dA)G (SEQ ID NO: 4); RVIRMCLNVGLLGEL(dA)G (SEQ ID NO: 5); RVIR(Nle)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNLGEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIRMCLGVGLLGDLAG (SEQ ID NO: 16); RVIRMCLGVGLLGDLAGK{PEG12} (SEQ ID NO: 17); {PEG12}KRVIRMCLNVGLLGEL(dA)E (SEQ ID NO: 18); RVIRMCLNVGLEGEL(dA) (SEQ ID NO: 19); RVIRMCLNVGLNGEL(dA)E (SEQ ID NO: 20); RVIRMCLNVGLNGE (SEQ ID NO: 21); RVIRMCLNNGLNGEL(dA)IG (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK{PEG12} (SEQ ID NO: 25); RVIRACLGVGLLGDL(dA)GK{PEG12} (SEQ ID NO: 29); RVIR(Nle)CLGVGLLGDL(dA)GK (SEQ ID NO: 33); RVIR(Nle)CLGVGLLGDL(dA)GK{PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLLGDLAGK{PEG12} (SEQ ID NO: 38); or pharmaceutically acceptable salts thereof.
229. The combination of any one of embodiments 224-227, wherein the peptide antifibrotic agent is selected from

```
(SEQ ID NO: 26)
RVIRMCLGVGLLGDLAG
          |
RVIRMCLGVGLLGDLAG;
or
(SEQ ID NO: 27)
RVIRMCLNVGLLGEL(dA)G
          |
RVIRMCLNVGLLGEL(dA)G;
or
(SEQ ID NO: 28)
RVIRMCLGVGLLGDLAGK{PEG12}
          |
RVIRMCLGVGLLGDLAGK{PEG12};
(SEQ ID NO: 30)
RVIRACLGVGLLGDL(dA)GK{PEG12}
          |
RVIRACLGVGLLGDL(dA)GK{PEG12};
and
(SEQ ID NO: 32)
RVIR(Nle)CLGVGLLGDL(dA)GK{PEG12}
                  |
RVIR(Nle)CLGVGLLGDL(dA)GK{PEG12}.
```

230. The combination of any one of embodiments 224-229, wherein the first antifibrotic agent comprises one or more agents selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-6, αVβ1/αVβ6 inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, IRE-1 inhibitors, P2X3 antagonists, SRC inhibitors, Jun N-terminal kinase (JNK) 1/2 inhibitors, JNK1 inhibitors, NOX 1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.
231. The combination of any one of embodiments 224-229, wherein the first antifibrotic agent comprises one or more agents selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-β inhibitors, anti-BAFF-R antibodies, calpain inhibitors, antibodies targeting integrin alpha-v beta-β, αVβ1/αVβ6 inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, Jun N-terminal kinase (JNK) 1/2 inhibitors, JNK1 inhibitors, NOX1/4 inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.
232. The combination of any one of embodiments 224-229, wherein the first antifibrotic agent comprises a PDGF receptor inhibitor or a TGF-β inhibitor.
233. The combination of embodiment 232, wherein the first antifibrotic agent comprises a TGF-β selected from vactosertib (TEW7197), pirfenidone and galunisertib.
234. The combination of embodiment 232, wherein the first antifibrotic agent comprises a PDGF receptor inhibitor selected from nintedanib, sunitinib, imatinib and sorafenib.
235. The combination of any one of embodiments 224-229, wherein the first antibibrotic agent comprises one or more agents selected from nintedanib, pirfenidone, indolinone, simtuzumab, (Gilead, GS-6624), IW001 (ImmuneWorks), PRM-151 (recombinant human pentraxin-2 protein, Promedior), tanzisertib (Celgene, CC-930), imatinib, STX-100 (Biogen), dectrekumab (Novartis, QAX576), pamrevlumab (FibroGen), carlumab (Janssen, CNTO-888), SM-04646 (Samumed), N-acetylcysteine (NAC), CC-90001 (BMS, Celgene), BMS-986,020 (Bristol Myers Squibb), BMS-986,278 (Bristol Myers Squibb), BBT-877 (Bridge Biotherapeutics and Boehringer Ingelheim), GLPG1690 (Galapagos), BI 1015550 (Boehringer Ingelheim), gefapixant, Setogepram (ProMetic, PBI-4050), tipelukast (MediciNova), GB 2064 (Galecto, PAT-1251), lanalumab (Novartis), BLD-2660 (Blade), PLN-74809 (Pliant), ND-L02-s0201 (Nitto Denko), setanaxib (GKT137831, Genkyotex), belumosudil (KD025, Kadmon), GB0139 (Galecto), BNC₁₀₂₁ (BONAC/Toray, TRK-250), ORIN1001 (Fosun), sildenafil, macitentan, bosentan, lebrikizumab, valganciclovir, letemovir, minocycline, zileuton, NIP292 (CR Pharma), voxelotor (GBT446), HEC_{825,} HEC_{6840,} and saracatinab.
236. The combination of any one of embodiments 224-229, wherein the first antifibrotic agent comprises one or more agents selected from pirfenidone or nintedanib.
237. The combination according to any one of embodiments 224-236 for use in the treatment of fibrosis in a patient in need of such treatment.
238. A peptide of Formula I comprising:

```
(SEQ ID NO: 32)
RVIR(Nle)CLGVGLLGDL(dA)GK{PEG12}
                  |
RVIR(Nle)CLGVGLLGDL(dA)GK{PEG12};
(SEQ ID NO: 33)
RVIR(Nle)CLGVGLLGDL(dA)GK;
(SEQ ID NO: 34)
RVIR(Nle)CLGVGLLGDL(dA)GK{PEG12};
(SEQ ID NO: 35)
RVIRACLGVGLLGDL(dA)GK;
(SEQ ID NO: 36)
RVIRACLGVGLLGDLAGK{PEG12}
          |
RVIRACLGVGLLGDLAGK{PEG12};
(SEQ ID NO: 37):
RVIRACLGVGLLGDLAGK
or
(SEQ ID NO: 38)
RVIRACLGVGLLGDLAGK{PEG12},
```

or an analog of said peptide, or derivative thereof, or pharmaceutically acceptable salt thereof.
239. A pharmaceutical composition comprising a peptide, analog, derivative, or pharmaceutically acceptable salt thereof of embodiment 238.
240. The peptide according to embodiment 236 or the pharmaceutical composition according to embodiment 237 for use in the treatment of fibrosis in a patient in need of such treatment.
241. A method for treating fibrosis in a patient in need of such treatment, the method comprising administering to the patient the peptide or derivative thereof, or pharmaceutically acceptable salt thereof according to embodiment 238, or administering a pharmaceutical composition comprising said peptide or derivative thereof or pharmaceutically acceptable salt thereof.

The embodiments described in the preceding paragraphs can alternatively he expressed as “Swiss use” embodiments.For example, the embodiments include:
401. Use of a first antifibrotic agent in the manufacture of a medicament, wherein the medicament is for treatment of fibrosis, for use in combination with a peptide antibrotic agent, or analog of said peptide, or derivative thereof, or pharmaceutically acceptable salt thereof, said peptide antifibrotic agent comprising an amino acid sequence of Formula I:

(SEQ ID NO: 31)
X¹-R-X²-IR-X³-X⁴-L-X⁵-X⁶-G-X¹⁴-X⁷-G-X⁸-X⁹ (I)

wherein X′ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK, -LAGK; or -L(dA); and X¹⁴ is N or L; or C-terminal acids or amides, or N-acetyl derivatives thereof; or pharmaceutically acceptable salts thereof.

The peptides and their uses having been described, the following examples are offered by of illustration, and not limitation.

### EXAMPLES

### Example 1

### Therapeutic Mouse Model of Idiopathic Pulmonary Fibrosis

The effect of combining the peptides of Formula I with a second antifibrotic drug on the progression of established lung fibrosis were assessed by monitoring lung fibrosis, lung weight, inflammatory cells in bronchoalveolar lavage fluid (BALF), soluble collagen in BALF, cytokine secretion, and body weight change in a therapeutic mouse model of idiopathic pulmonary fibrosis. Lung fibrosis was induced in lungs of male C₅₇BL/6 mice between the ages of 6 to 8 weeks by nasophayngeal administration of bleomycin (1.5U/kg using bleomycin clinical formulation diluted in PBS). A control group of animals were administered saline by the nasopharyngeal route (no-bleomycin control group). After one week, bleomycin-treated animals were randomized to treatment groups (N=10 per group) by body weight and treated daily with vehicle control, nintedanib (60 mg/kg/day PO), peptide (5 mg/kg/day by intraperitoneal injection), or the combination of nintedanib (60 mg/kg/day PO) and peptide (5 mg/kg/day by intraperitoneal injection). After 14 days of treatment (Day 21), lungs were removed and weighed. The post-caval lobe was separated and snap frozen. The lungs were flushed with Hanks Buffer and bronchoalveolar lavage fluid (BALF) was harvested from each animal Differential cell counts in BALF were obtained. Levels of pro-inflammatory cytokines in BALF were determined using a meso scale discovery (MSD) system. Slides were prepared from the remaining BALF leukocytes, fixed and stained with May Geimsa stain and the differential counts were recorded manually. BALF was evaluated for soluble collagen using a Sircol assay. Lungs were fixed in 10% neutral buffered formalin (NBF) for histopathological analysis. Fibrosis was assessed by histopathological analysis of H&E stained mouse lungs using an Ashcroft scoring system. Collagen deposition in lung tissue was assessed by histopathological analysis of Masson's Trichrome stained mouse lungs using a standard scoring system for severity. Table 4 shows that treatment with a combination of SEQ ID NO: 32 and nintedanib had a greater effect that treatment with either compound alone on reducing lung weight, collagen secretion in BALF, collagen deposition in lung tissue, and lung fibrosis. Table 5 shows that treatment with a combination of SEQ ID NO: 32 and nintedanib had a greater effect than treatment with either compound alone on reducing inflammation based on decrease in numbers of macrophages, lymphocytes, and neutrophils in BALF. Table 6 shows that treatment with a combination of SEQ ID NO: 32 and nintedanib had a greater effect than treatment with either compound alone on reducing secretion of key pro-inflammatory cytokines in BALF.

TABLE 4
Efficacy Parameters in a Therapeutic Mouse Model of Idiopathic Pulmonary Fibrosis

| | | Mean (SEM) | | |
|---|---|---|---|---|
| | | Soluble | Mean (SEM) | |
| | | Collagen | Collagen | |
| | Mean (SEM) | Content in | Deposition | Mean (SEM) |
| | Normalized | BALF | Score in | Ashcroft |
| Treatment | Lung Weight | (mg/mL) | Lung Tissue | Score |
| No Bleomycin | 0.87 (0.02) | 0.018 (0.001) | 0.00 (0.00) | 0.00 (0.00) |
| Bleomycin/Vehicle | 1.95 (0.17) | 0.092 (0.010) | 3.70 (0.26) | 4.08 (0.26) |
| Bleomycin/Nintedanib | 1.49 (0.17) | 0.048 (0.005) | 2.22 (0.43) | 2.82 (0.39) |
| Bleomycin/SEQ ID NO: 32 | 1.57 (0.20) | 0.058 (0.005) | 2.56 (0.48) | 3.12 (0.45) |
| Bleomycin/Nintedanib + SEQ ID NO: 32 | 1.16 (0.05) | 0.038 (0.004) | 1.75 (0.25) | 2.07 (0.34) |
| Bleomycin/SEQ ID NO: 30 | 1.36 (0.10) | 0.067 (0.013) | 2.30 (0.26) | 2.93 (0.33) |
| Bleomycin/ | 1.44 (0.10) | 0.041 (0.005) | 2.50 (0.31) | 3.00 (0.28) |
| Nintedanib + SEQ ID NO: 30 | | | | |

TABLE 5
Differential Blood Cell Counts in Bronchoalveolar Lavage Fluid in a Therapeutic Mouse
Model of Idiopathic Pulmonary Fibrosis

| | Mean (SEM) | Mean (SEM) | Mean (SEM) |
|---|---|---|---|
| | Number of | Number of | Number of |
| | Macrophages | Lymphocytes | Neutrophils |
| Treatment | in BALF | in BALF | in BALF |
| No Bleomycin | 6060 (959) | 0 (0) | 154 (103) |
| Bleomycin/ | 95300 (14400) | 33500 (9760) | 9100 (3060) |
| Vehicle | | | |
| Bleomycin/ | 49200 (6450) | 18700 (5200) | 3040 (1370) |
| Nintedanib | | | |
| Bleomycin/ | 86600 (7070) | 28600 (5480) | 5850 (2220) |
| SEQ ID NO: 32 | | | |
| Bleomycin/ | 34500 (6010) | 2060 (1400) | 1270 (1270) |
| Nintedanib + | | | |
| SEQ ID NO: 32 | | | |
| Bleomycin/ | 41700 (5740) | 8930 (3350) | 1830 (1080) |
| SEQ ID NO: 30 | | | |
| Bleomycin/ | 44100 (6810) | 7090 (2640) | 3500 (1530) |
| Nintedanib + | | | |
| SEQ ID NO: 30 | | | |

TABLE 6
Pro-inflammatory Cytokine Levels in Bronchoalveolar Lavage Fluid in a Therapeutic
Mouse Model of Idiopathic Pulmonary Fibrosis

| | Mean (SEM) Pro-inflammatory Cytokine Level (pg/mL) | | | | | | |
|---|---|---|---|---|---|---|---|
| Treatment* | TNFα | IL-1β | IFNγ | KC/GRO | IL-6 | MCP-1 | MIP-1α |
| No Bleomycin | 0.25 | 0.37 | 0.00 | 4.75 | 0.21 | 0.66 | 0.30 |
| | (0.05) | (0.19) | (0.00) | (0.74) | (0.19) | (0.40) | (0.08) |
| Bleomycin/ | 4.52 | 0.36 | 0.022 | 29.2 | 239 | 34.4 | 1.67 |
| Vehicle | (0.87) | (0.10) | (0.011) | (6.84) | (67.6) | (10.9) | (0.30) |
| Bleomycin/ | 2.76 | 0.50 | 0.019 | 29.3 | 384 | 35.8 | 1.05 |
| Nintedanib | (0.30) | (0.14) | (0.014) | (6.84) | (177) | (17.1) | (0.22) |
| Bleomycin/ | 3.05 | 0.07 | 0.020 | 21.5 | 269 | 29.7 | 0.95 |
| SEQ ID NO: 32 | (0.26) | (0.07) | (0.010) | (2.80) | (152) | (16.6) | (0.25) |
| Bleomycin/ | 1.36 | 0.00 | 0.010 | 10.1 | 8.63 | 1.97 | 0.26 |
| Nintedanib + | (0.39) | (0.00) | (0.010) | (1.72) | (4.77) | (0.98) | (0.08) |
| SEQ ID NO: 32 | | | | | | | |
| Bleomycin/ | 2.85 | 0.03 | 0.037 | 16.7 | 64.6 | 7.59 | 0.52 |
| SEQ ID NO: 30 | (0.56) | (0.03) | (0.032) | (3.10) | (32.1) | (3.00) | (0.11) |
| Bleomycin/ | 2.47 | 0.03 | 0.036 | 14.4 | 58.7 | 10.7 | 0.50 |
| Nintedanib + | (0.36) | (0.03) | (0.033) | (1.51) | (26.6) | (3.79) | (0.10) |
| SEQ ID NO: 30 | | | | | | | |

### Example 2

### Therapeutic Mouse Model of Idiopathic Pulmonary Fibrosis

The effect of the peptides on the progression of established lung fibrosis were assessed by monitoring lung fibrosis, lung weight, inflammatory cells in bronchoalveolar lavage fluid (BALF), soluble collagen in BALF, cytokine secretion in BALF, and body weight change in a therapeutic mouse model of idiopathic pulmonary fibrosis. Lung fibrosis was induced in lungs of male C57BL/6 mice between the ages of 6 to 8 weeks by nasophayngeal administration of bleomycin (1.5 U/kg using bleomycin clinical formulation diluted in PBS). A control group of animals were administered saline by the nasopharyngeal route (no-bleomycin control group). After one week, bleomycin-treated animals were randomized to treatment groups (N=10 per group) by body weight and treated daily with vehicle control, nintedanib positive control (60 mg/kg/day PO) or peptide (5 or 15 mg/kg/day by intraperitoneal injection). After 14 days of treatment (Day 21), lungs were removed and weighed. The post-caval lobe was separated and snap frozen. The lungs were flushed with Hanks Buffer and bronchoalveolar lavage fluid (BALF) was harvested from each animal Total BALF leukocyte were counted. Slides were prepared from the remaining BALF leukocytes, fixed and stained with May Geimsa stain and the differential counts were recorded manually. BALF was evaluated for soluble collagen using a Sircol assay. Lungs were fixed in 10% neutral buffered formalin (NBF) for histopathological analysis. Fibrosis was assessed by histopathological analysis of H&E stained mouse lungs using an Ashcroft scoring system. Collagen deposition in lung tissue was assessed by histopathological analysis of Masson's Trichrome stained mouse lungs using a standard scoring system for severity.

TABLE 7
Efficacy Parameters in Therapeutic Mouse Model of Idiopathic Pulmonary Fibrosis

| | | Mean | Mean (SEM) | Mean (SEM) | |
|---|---|---|---|---|---|
| | | (SEM) | Collagen | Collagen | Mean |
| | Dose | Normalized | Deposition | Content in | (SEM) |
| | (mg/kg/ | Lung | Score in Lung | BALF | Ashcroft |
| Treatment | day) | Weight | Tissue | (mg/mL) | Score |
| No Bleomycin | N/A | 0.90 (0.01) | 0.00 (0.00) | 0.010 (0.001) | 0.00 (0.00) |
| Bleomycin/Vehicle | N/A | 1.57 (0.07) | 3.10 (0.28 | 0.122 (0.013) | 3.51 (0.24) |
| Bleomycin/Nintedanib | 60 | 1.16 (0.07) | 2.20 (0.36) | 0.061 (0.011) | 2.35 (0.38) |
| Bleomycin/SEQ ID NO: 28 | 15 | 1.27 (0.05) | 2.00 (0.26) | 0.080 (0.016) | 2.24 (0.25) |
| Bleomycin/ | 5 | 1.17 (0.03) | 2.20 (0.25) | 0.074 (0.019) | 2.26 (0.27) |
| SEQ ID NO: 32 | | | | | |
| Bleomycin/ | 15 | 1.20 (0.05) | 2.10 (0.35) | 0.091 (0.016) | 2.26 (0.35) |
| SEQ ID NO: 32 | | | | | |
| Bleomycin/ | 5 | 1.15 (0.03) | 1.67 (0.29) | 0.071 (0.020) | 1.92 (0.21) |
| SEQ ID NO: 30 | | | | | |
| Bleomycin/ | 15 | 1.22 (0.11) | 1.80 (0.33) | 0.063 (0.007) | 2.00 (0.37) |
| SEQ ID NO: 30 | | | | | |
| Bleomycin/ | 5 | 1.19 (0.06) | 2.50 (0.37) | 0.081 (0.010) | 2.65 (0.40) |
| SEQ ID NO: 36 | | | | | |
| Bleomycin/ | 15 | 1.32 (0.12) | 2.89 (0.35) | 0.068 (0.008) | 3.33 (0.33) |
| SEQ ID NO: 36 | | | | | |

TABLE 8
Diferential Cell Counts in BALF in Therapeutic Mouse Model of Idiopathic Pulmonary Fibrosis

| | | Mean (SEM) | Mean (SEM) | Mean (SEM) |
|---|---|---|---|---|
| | | Number of | Number of | Number of |
| | Dose | Macrophages | Lymphocytes | Neutrophils |
| Treatment | (mg/kg/day) | in BALF | in BALF | in BALF |
| No Bleomycin | N/A | 328 (40) | 0 (0) | 10 (7) |
| Bleomycin/ | N/A | 9110 (965) | 2105 (460) | 659 (171) |
| Vehicle | | | | |
| Bleomycin/ | 60 | 5030 (823) | 1180 (631) | 201 (77) |
| Nintedanib | | | | |
| Bleomycin/ | 15 | 6340 (792) | 1210 (233) | 261 (117) |
| SEQ ID NO: 28 | | | | |
| Bleomycin/ | 5 | 5530 (1090) | 946 (579) | 61 (61) |
| SEQ ID NO: 32 | | | | |
| Bleomycin/ | 15 | 5030 (974) | 1270 (584) | 96 (64) |
| SEQ ID NO: 32 | | | | |
| Bleomycin/ | 5 | 5070 (1130) | 1210 (664) | 0 (0) |
| SEQ ID NO: 30 | | | | |
| Bleomycin/ | 15 | 3780 (559) | 896 (254) | 122 (88) |
| SEQ ID NO: 30 | | | | |
| Bleomycin/ | 5 | 5180 (1030) | 987 (316) | 113 (60) |
| SEQ ID NO: 36 | | | | |
| Bleomycin/ | 15 | 6430 (930) | 884 (442) | 165 (140) |
| SEQ ID NO: 36 | | | | |

### Example 3 (Prophetic)

### Therapeutic Mouse Model of Idiopathic Pulmonary Fibrosis

The effect of combining the peptides of Formula I with a second antifibrotic drug on the progression of established lung fibrosis can be assessed by monitoring lung fibrosis, lung weight, inflammatory cells in bronchoalveolar lavage fluid (BALF), soluble collagen in BALF, cytokine secretion, and body weight change in a therapeutic mouse model of idiopathic pulmonary fibrosis. Lung fibrosis is induced in lungs of male C₅₇BL/6 mice between the ages of 6 to 8 weeks by nasophayngeal administration of bleomycin (1.5U/kg using bleomycin clinical formulation diluted in PBS). A control group of animals are administered saline by the nasopharyngeal route (no-bleomycin control group). After one week, bleomycin-treated animals are randomized to treatment groups (N=10 per group) by body weight and treated daily with vehicle control, a first antifibrotic agent (60 mg/kg/day PO), peptide (5 mg/kg/day by intraperitoneal injection), or the combination of a first antifibrotic agent (60 mg/kg/day PO) and peptide (5 mg/kg/day by intraperitoneal injection).

After 14 days of treatment (Day 21), lungs are removed and weighed. The post-caval lobe is separated and snap frozen. The lungs are flushed with Hanks Buffer and bronchoalveolar lavage fluid (BALF) is harvested from each animal. Differential cell counts in BALF are obtained. Levels of pro-inflammatory cytokines in BALF are determined using a meso scale discovery (MSD) system. Slides are prepared from the remaining BALF leukocytes, fixed and stained with May Geimsa stain and the differential counts were recorded manually. BALF is evaluated for soluble collagen using a Sircol assay. Lungs are fixed in 10% neutral buffered formalin (NBF) for histopathological analysis. Fibrosis is assessed by histopathological analysis of H&E stained mouse lungs using an Ashcroft scoring system. Collagen deposition in lung tissue is assessed by histopathological analysis of Masson's Trichrome stained mouse lungs using a standard scoring system for severity. Evidence that treatment with a combination of the peptide antifibrotic agent and the first antifibrotic agent have a greater effect than treatment with either compound alone on reducing one or more indicia of fibrosis, including lung weight, collagen secretion in BALF, collagen deposition in lung tissue, and lung fibrosis, is indicative of an efficacious combination.

In a variation of this protocol, doses of the peptide antifibrotic agent and/or the first antifibrotic agent are varied. Evidence that a threshold beneficial antifibrotic effect is achieved with a lower dose of the first antifibrotic agent when used in combination with the peptide is indicative of an efficacious combination.

TABLE 9
Combinations Active in Therapeutic Mouse
Model of Idiopathic Pulmonary Fibrosis

| First Antifibrotic Agent | Peptide |
|---|---|
| PRM-151 (recombinant human | SEQ ID NO: 30 |
| pentraxin-2 protein) | |
| BI 1015550 | SEQ ID NO: 32 |
| GB0139 | SEQ ID NO: 32 |
| CC-90001 | SEQ ID NO: 30 |
| Pamrevlumab | SEQ ID NO: 30 |

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

For conciseness, several aspects or embodiments are described herein as genera and/or as lists of alternative species. In each instance, subgenera and individual species are contemplated as individual aspects or embodiments of the invention.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as being limiting in any way. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. The citation and incorporation of patent documents herein is done for convenience only and does not reflect any view of the validity, patentability, and/or enforceability of such patent documents.

This disclosure includes all modifications and equivalents of the subject matter recited in the aspects appended hereto as permitted by applicable law.

1. A method for treating fibrosis in a patient in need of such treatment, the method comprising administering to the patient a pharmacologically effective amount of a first antifibrotic agent and a peptide antibrotic agent, or analog of said peptide, or derivative thereof, or pharmaceutically acceptable salt thereof,
said peptide antifibrotic agent comprising an amino acid sequence of Formula I:
(SEQ ID NO: 31)
X¹-R-X²-IR-X³-X⁴-L-X⁵-X⁶-G-X¹⁴-X⁷-G-X⁸-X⁹ (I)
wherein X¹ is absent, K or M; X² is V or d(A); X³ is M, A or Nle; X⁴ is C or S; X⁵ is G or N; X⁶ is V or N; X⁷ is L, N or E; X⁸ is D or E; X⁹ is absent, -LAG, -L(dA)G, -L(dA)E, -L(dA)GK , -LAGK; or -L(dA); and X¹⁴ is N or L; or C-terminal acids or amides, or N-acetyl derivatives thereof; or pharmaceutically acceptable salts thereof.

2. (canceled)

3. The method of claim 1, wherein the fibrosis is any of cirrhosis of the liver; pulmonary fibrosis, idiopathic pulmonary fibrosis; fibrosis following myocardial infarction; CNS fibrosis following a stroke, neurodegenerative disorders, Alzheimer's Disease, multiple sclerosis; proliferative vitreoretinopathy (PVR), arthritis; adhesions, nephrogenic systemic fibrosis; myocardial fibrosis; hepatic fibrosis; epidural fibrosis (failed back surgery syndrome); endomyocardial fibrosis; tubulointerstitial fibrosis; renal interstitial fibrosis; mediastinal fibrosis; retroperitoneal fibrosis; penile fibrosis; oral submucous; kidney fibrosis; idiopathic pulmonary upper lobe fibrosis; congenital hepatic fibrosis; postlaminotomy fibrosis; painful disc fibrosis; graft fibrosis; atrial fibrosis; corneal subepithelial fibrosis; congenital orbital fibrosis; bone fibrosis; peritoneal fibrosis; nephrogenic systemic fibrosis; non-cirrhotic portal fibrosis; pulmonary tuberculosis, disease-related pulmonary apical fibrosis in ankylosing spondylitis; colorectal fibrosis; periglomerular fibrosislatubular glomeruli; basal fibrosis syndrome; tissue fibrosis; and massive neck fibrosis.

4. The method of claim 1, wherein the fibrosis is idiopathic pulmonary fibrosis.

5. The method of claim 1, wherein the fibrosis is scleroderma or systemic sclerosis.

6. The method of claim 1, wherein the peptide antifibrotic agent comprises an amino acid sequence selected from MRVIRMCLGVGLLGDLAG (SEQ ID NO: 2); RVIRMCLGVGLLGDLAG (SEQ ID NO: 3); RVIRMCLGVGLLGDL(dA)G (SEQ ID NO: 4); RVIRMCLNVGLLGEL(dA)G (SEQ ID NO: 5); RVIR(Nle)CLNVGLLGEL(dA)G (SEQ ID NO: 6); RVIRMSLNVGLLGEL(dA)G (SEQ ID NO: 7); RVIR(Nle)SLNVGLLGEL(dA)G (SEQ ID NO: 8); RVIRMCLNNGLLGEL(dA)G (SEQ ID NO: 9); RVIRMCLNVGNLGEL(dA)G (SEQ ID NO: 10); RVIRMCLNVGLNGEL(dA)G (SEQ ID NO: 11); RVIRMCLNVGLLGEL(dA)E (SEQ ID NO: 12); RVIRMSLNVGLEGEL(dA) (SEQ ID NO: 13); RVIR(Nle)SLNVGLEGEL(dA) (SEQ ID NO: 14); R(dA)IR(Nle)SLNVGLLGEL(dA) (SEQ ID NO: 15); {PEG12}KRVIRMCLGVGLLGDLAG (SEQ ID NO: 16); RVIRMCLGVGLLGDLAGK{PEG12} (SEQ ID NO: 17); {PEG12}KRVIRMCLNVGLLGEL(dA)E (SEQ ID NO: 18); RVIRMCLNVGLEGEL(dA) (SEQ ID NO: 19); RVIRMCLNVGLNGEL(dA)E (SEQ ID NO: 20); RVIRMCLNVGLNGE (SEQ ID NO: 21); RVIRMCLNNGLNGEL(dA)}G (SEQ ID NO: 22); RVIRMCLNNGLNGEL(dA)E (SEQ ID NO: 23); {5-FAM}-RVIRMCLGVGLLGDLAG (SEQ ID NO: 24); {5-FAM}-RVIRMCLGVGLLGDLAGK{PEG12} (SEQ ID NO: 25); RVIRACLGVGLLGDL(dA)GK{PEG12} (SEQ ID NO: 29); RVIR(Nle)CLGVGLLGDL(dA)GK (SEQ ID NO: 33); RVIR(Nle)CLGVGLLGDL(dA)GK{PEG12} (SEQ ID NO: 34); RVIRACLGVGLLGDL(dA)GK (SEQ ID NO: 35) RVIRACLGVGLLGDLAGK (SEQ ID NO: 37); and RVIRACLGVGLLGDLAGK{PEG12} (SEQ ID NO: 38); or pharmaceutically acceptable salts thereof.

7. The method of claim 1, wherein the peptide antifibrotic agent comprises a peptide dimer comprised of peptides comprising an amino acid sequence of Formula I.

8. The method of claim 1, wherein the peptide antifibrotic agent comprises a peptide or dimer thereof that is derivatized via acetylation, pegylation, biotinylation or acylation.

9. The method of claim 1 wherein the peptide antifibrotic agent is selected from
```
(SEQ ID NO: 26)
RVIRMCLGVGLLGDLAG
          |
RVIRMCLGVGLLGDLAG;
(SEQ ID NO: 27)
RVIRMCLNVGLLGEL(dA)G
          |
RVIRMCLNVGLLGEL(dA)G;
(SEQ ID NO: 28)
RVIRMCLGVGLLGDLAGK{PEG12}
          |
RVIRMCLGVGLLGDLAGK{PEG12};
(SEQ ID NO: 30)
RVIRACLGVGLLGDL(dA)GK{PEG12}
          |
RVIRACLGVGLLGDL(dA)GK{PEG12};
(SEQ ID NO: 32)
RVIR(Nle)CLGVGLLGDL(dA)GK{PEG12}
                  |
RVIR(Nle)CLGVGLLGDL(dA)GK{PEG12};
(SEQ ID NO: 33)
RVIR(Nle)CLGVGLLGDL(dA)GK;
(SEQ ID NO: 34)
RVIR(Nle)CLGVGLLGDL(dA)GK{PEG12};
(SEQ ID NO: 35)
RVIRACLGVGLLGDL(dA)GK;
(SEQ ID NO: 36)
RVIRACLGVGLLGDLAGK{PEG12}
          |
RVIRACLGVGLLGDLAGK{PEG12};
(SEQ ID NO: 37)
RVIRACLGVGLLGDLAGK;
or
(SEQ ID NO: 38)
RVIRACLGVGLLGDLAGK{PEG12}.
```

10. (canceled)

11. The method of claim 1, wherein the first antifibrotic agent comprises one or more agents selected from anti-LOXL2 antibodies, recombinant pentraxins, PDGF inhibitors, PDGF receptor inhibitors, FGF receptor inhibitors, vascular endothelial growth factor receptor (VEGFR) inhibitors, TGF inhibitors, TGF-6 inhibitors, anti-BAFF-R antibodies, calpain inhibitors, antibodies targeting integrin alpha-v beta-6, αᵥβ₁/αᵥβ₆ inhibitors, antibodies targeting IL-13, antibodies targeting CTGF, antibodies targeting CCL2, anti-CCN2 antibodies, lysophospholipid receptor (LPA1) antagonists, glutamate 2b receptor antagonists, WNT/MET inhibitors, N-acetylcysteine (NAC; anti-oxidant), MK-2 inhibitors, Heat Shock Protein 47 (HSP47) gene therapies, IRE-1 inhibitors, P2X3 antagonists, SRC inhibitors, Jun N-terminal kinase (JNK) 1/2 inhibitors, JNK1 inhibitors, NOX^{1/4} inhibitors, autotaxin inhibitors, endoplasmic reticulum stress (ER stress) inhibitors, galectin-3 inhibitors, leukotriene inhibitors, leukotriene (LT) receptor antagonists, phosphodiesterases (PDE) inhibitors, 5-lipoxygenase (5-LO) inhibitors, Rho-associated kinase (ROCK2) inhibitors, and LoxL2 inhibitors.

12. (canceled)

13. The method of claim 1, wherein the first antifibrotic agent comprises a PDGF receptor inhibitor or a TGF-β inhibitor.

14. The method of claim 13, wherein the first antifibrotic agent comprises a TGF-β inhibitor selected from vactosertib (TEW7197), pirfenidone, and galunisertib or a PDGF receptor inhibitor selected from nintedanib, sunitinib, imatinib, and sorafenib.

15. (canceled)

16. The method of claim 1, wherein the first antifibrotic agent comprises one or more agents selected from nintedanib, pirfenidone, indolinone, simtuzumab, (Gilead, GS-6624), IW001 (ImmuneWorks), PRM-151 (recombinant human pentraxin-2 protein, Genetech, Promedior), TANZISERTIB (Celgene, CC-930), imatinib, STX-100 (Biogen), dectrekumab (Novartis, QAX576), pamrevlumab (FibroGen), carlumab (Janssen, CNTO-888), SM-04646 (Samumed), N-acetylcysteine (NAC), CC-90001 (Celgene), BMS-986,020 (Bristol Myers Squibb), BMS-986,278 (Bristol Myers Squibb), BBT-877 (Bridge Biotherapeutics and Boehringer Ingelheim), GLPG1690 (Galapagos), BI 1015550 (Boehringer Ingelheim), Gefapixant, Setogepram (ProMetic, PBI-4050), tipelukast (MediciNova), GB 2064 (GALECTO, PAT-1251), lanalumab (Novartis), BLD-2660 (Blade), PLN-74809 (Pliant), ND-L02-50201 (Nitto Denko), setanaxib (GKT137831, Genkyotex), belumosudil (KD025, Kadmon), GB0139 (Galecto), BNC₁₀₂₁ (BONAC/Toray, TRK-250), ORIN1001 (Fosun), sildenafil, macitentan, bosentan, lebrikizumab, valganciclovir, letemovir, minocycline, gefapixant, zileuton, NIP292 (CR Pharma), voxelotor (GBT446), HEC_{825,} HEC_{6840,} saracatinab, and CC₉₀₀₀₁ (BMS).

17.-18. (canceled)

19. The method of claim 1, wherein the first antifibrotic agent comprises one or more agents selected from pirfenidone and nintedanib.

20. (canceled)

21. The method of claim 1 that comprises administering the first antifibrotic agent and the peptide antifibrotic agent simultaneously.

22. The method of claim 1 that comprises administering the first antifibrotic agent and the peptide antifibrotic agent sequentially.

23.-37. (canceled)

38. A peptide of Formula I comprising:
```
(SEQ ID NO: 32)
RVIR(Nle)CLGVGLLGDL(dA)GK{PEG12}
                  |
RVIR(Nle)CLGVGLLGDL(dA)GK{PEG12};
(SEQ ID NO: 33)
RVIR(Nle)CLGVGLLGDL(dA)GK;
(SEQ ID NO: 34)
RVIR(Nle)CLGVGLLGDL(dA)GK{PEG12};
(SEQ ID NO: 35)
RVIRACLGVGLLGDL(dA)GK;
(SEQ ID NO: 36)
RVIRACLGVGLLGDLAGK {PEG12}
          |
RVIRACLGVGLLGDLAGK{PEG12};
(SEQ ID NO: 37):
RVIRACLGVGLLGDLAGK
or
(SEQ ID NO: 38)
RVIRACLGVGLLGDLAGK{PEG12},
```
or an analog of said peptide, or derivative thereof, or pharmaceutically acceptable salt thereof.

39. A pharmaceutical composition comprising a peptide, analog, derivative, or pharmaceutically acceptable salt thereof of claim 38.

40. (canceled)

41. A method for treating fibrosis in a patient in need of such treatment, the method comprising administering to the patient the peptide or derivative thereof, or pharmaceutically acceptable salt thereof according to claim 38, or administering a pharmaceutical composition comprising said peptide or derivative thereof or pharmaceutically acceptable salt thereof.


SEQUENCE LISTING (42 sequences)

SEQ ID NO: 1 - protein, 19 aa, Artificial Sequence
  Synthetic Polypeptide
  MISC_FEATURE (1)..(1): Xaa is absent or an amino acid having a polar or non-polar side chain
  MISC_FEATURE (1)..(1): Xaa can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys, Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (3)..(3): Xaa is an amino acid having a non-polar side chain
  MISC_FEATURE (3)..(3): Xaa can be Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (6)..(6): Xaa is an amino acid having a non-polar side chain
  MISC_FEATURE (6)..(6): Xaa can be Gly, Ala, D-Ala, Val, D-Val, Leu, D-Leu, Ile, D-Ile, Phe, D-Phe, Trp, D-Trp, Pro, D-Pro, Nle, Met, and D-Met
  MISC_FEATURE (7)..(7): Xaa is an amino acid having a polar side chain
  MISC_FEATURE (7)..(7): Xaa can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys
  MISC_FEATURE (9)..(9): Xaa is an amino acid having polar or non-polar side chain
  MISC_FEATURE (9)..(9): Xaa can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys, Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (10)..(10): Xaa is an amino acid having polar or non-polar side chain
  MISC_FEATURE (10)..(10): Xaa can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys, Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (13)..(13): Xaa is an amino acid having polar or non-polar side chain
  MISC_FEATURE (13)..(13): Xaa can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys, Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (15)..(15): Xaa is an amino acid having polar side chain
  MISC_FEATURE (15)..(15): Xaa can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys
  MISC_FEATURE (16)..(16): Xaa is absent or an amino acid having a non- polar side chain
  MISC_FEATURE (16)..(16): Xaa, when present, can be Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (17)..(17): Xaa is absent if  Xaa16 is absent; if Xaa16 is present, Xaa is an amino acid having a non-polar sidechain
  MISC_FEATURE (17)..(17): Xaa, when present, can be Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (18)..(18): Xaa is absent if Xaa16 is absent; if Xaa16 is present, Xaa is absent or is an amino acid having a polar or non- polar side chain
  MISC_FEATURE (18)..(18): Xaa, when present, can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys, Gly, Ala, D-Ala, Val, D-Val, Leu, D-Leu, Ile, D-Ile, Phe, D-Phe, Trp, D-Trp, Pro, D-Pro, Met, D-Met
  MISC_FEATURE (19)..(19): Xaa is absent if Xaa16 or Xaa18 is absent; if Xaa16 and Xaa18 are both present, then Xaa is absent or is an amino acid having a polar side chain
  MISC_FEATURE (19)..(19): Xaa, when present, can be Asp, D-Asp, Glu, D-Glu, Lys, D-Lys, Arg, D-Arg, His, D-His, Asn, D-Asn, Gln, D-Gln, Ser, D-Ser, Thr, D-Thr, Tyr, D-Tyr, Cys, D-Cys
XRXIRXXLXXGLXGXXXXX

SEQ ID NO: 2 - protein, 18 aa, Artificial Sequence
  Synthetic Polypeptide
MRVIRMCLGVGLLGDLAG

SEQ ID NO: 3 - protein, 17 aa, Artificial Sequence
  Synthetic Polypeptide
RVIRMCLGVGLLGDLAG

SEQ ID NO: 4 - protein, 17 aa, Artificial Sequence
  Synthetic Polypeptide
  MISC_FEATURE (16)..(16): D-Ala
RVIRMCLGVGLLGDLAG

SEQ ID NO: 5 - protein, 17 aa, Artificial Sequence
  Synthetic Polypeptide
  MISC_FEATURE (16)..(16): D-Ala
RVIRMCLNVGLLGELAG

SEQ ID NO: 6 - protein, 17 aa, Artificial Sequence
  Synthetic Polypeptide
  MISC_FEATURE (5)..(5): Nle
  MISC_FEATURE (16)..(16): D-Ala
RVIRLCLNVGLLGELAG

SEQ ID NO: 7 - protein, 17 aa, Artificial Sequence
  Synthetic Polypeptide
  MISC_FEATURE (16)..(16): D-Ala
RVIRMSLNVGLLGELAG

SEQ ID NO: 8 - protein, 17 aa, Artificial Sequence
  Synthetic Polypeptide
  MISC_FEATURE (5)..(5): Nle
  MISC_FEATURE (16)..(16): D-Ala
RVIRLSLNVGLLGELAG

SEQ ID NO: 9 - protein, 17 aa, Artificial Sequence
  Synthetic Polypeptide
  MISC_FEATURE (16)..(16): D-Ala
RVIRMCLNNGLLGELAG

SEQ ID NO: 10 - protein, 17 aa, Artificial Sequence
  Synthetic Polypeptide
  MISC_FEATURE (16)..(16): D-Ala
RVIRMCLNVGNLGELAG

SEQ ID NO: 11 - protein, 17 aa, Artificial Sequence
  Synthetic Polypeptide
  MISC_FEATURE (16)..(16): D-Ala
RVIRMCLNVGLNGELAG

SEQ ID NO: 12 - protein, 17 aa, Artificial Sequence
  Synthetic Polypeptide
  MISC_FEATURE (16)..(16): D-Ala
RVIRMCLNVGLLGELAE

SEQ ID NO: 13 - protein, 16 aa, Artificial Sequence
  Synthetic Polypeptide
  MISC_FEATURE (16)..(16): D-Ala
RVIRMSLNVGLEGELA

SEQ ID NO: 14 - protein, 16 aa, Artificial Sequence
  Synthetic Polypeptide
  MISC_FEATURE (5)..(5): Nle
  MISC_FEATURE (16)..(16): D-Ala
RVIRLSLNVGLEGELA

SEQ ID NO: 15 - protein, 16 aa, Artificial Sequence
  Synthetic Polypeptide
  MISC_FEATURE (2)..(2): D-Ala
  MISC_FEATURE (5)..(5): Nle
  MISC_FEATURE (16)..(16): D-Ala
RAIRLSLNVGLLGELA

SEQ ID NO: 16 - protein, 18 aa, Artificial Sequence
  Synthetic Polypeptide
  MISC_FEATURE (1)..(1): PEG12
KRVIRMCLGVGLLGDLAG

SEQ ID NO: 17 - protein, 18 aa, Artificial Sequence
  Synthetic Polypeptide
  MISC_FEATURE (18)..(18): PEG12
RVIRMCLGVGLLGDLAGK

SEQ ID NO: 18 - protein, 18 aa, Artificial Sequence
  Synthetic Polypeptide
  MISC_FEATURE (1)..(1): PEG12
  MISC_FEATURE (17)..(17): D-Ala
KRVIRMCLNVGLLGELAE

SEQ ID NO: 19 - protein, 16 aa, Artificial Sequence
  Synthetic Polypeptide
  MISC_FEATURE (16)..(16): D-Ala
RVIRMCLNVGLEGELA

SEQ ID NO: 20 - protein, 17 aa, Artificial Sequence
  Synthetic Polypeptide
  MISC_FEATURE (16)..(16): D-Ala
RVIRMCLNVGLNGELAE

SEQ ID NO: 21 - protein, 14 aa, Artificial Sequence
  Synthetic Polypeptide
RVIRMCLNVGLNGE

SEQ ID NO: 22 - protein, 17 aa, Artificial Sequence
  Synthetic Polypeptide
  MISC_FEATURE (16)..(16): D-Ala
RVIRMCLNNGLNGELAG

SEQ ID NO: 23 - protein, 17 aa, Artificial Sequence
  Synthetic Polypeptide
  MISC_FEATURE (16)..(16): D-Ala
RVIRMCLNNGLNGELAE

SEQ ID NO: 24 - protein, 17 aa, Artificial Sequence
  Synthetic Polypeptide
  MISC_FEATURE (1)..(1): 5-FAM
RVIRMCLGVGLLGDLAG

SEQ ID NO: 25 - protein, 18 aa, Artificial Sequence
  Synthetic Polypeptide
  MISC_FEATURE (1)..(1): 5-FAM
  MISC_FEATURE (18)..(18): PEG12
RVIRMCLGVGLLGDLAGK

SEQ ID NO: 26 - protein, 17 aa, Artificial Sequence
  Synthetic Polypeptide
  DISULFID (6)..(6)
RVIRMCLGVGLLGDLAG

SEQ ID NO: 27 - protein, 17 aa, Artificial Sequence
  Synthetic Polypeptide
  DISULFID (6)..(6)
  MISC_FEATURE (16)..(16): D-Ala
RVIRMCLNVGLLGELAG

SEQ ID NO: 28 - protein, 18 aa, Artificial Sequence
  Synthetic Polypeptide
  DISULFID (6)..(6)
  MISC_FEATURE (18)..(18): PEG12
RVIRMCLGVGLLGDLAGK

SEQ ID NO: 29 - protein, 18 aa, Artificial Sequence
  Synthetic Polypeptide
  MISC_FEATURE (16)..(16): d-Ala
  MISC_FEATURE (18)..(18): PEG12
RVIRACLGVGLLGDLAGK

SEQ ID NO: 30 - protein, 18 aa, Artificial Sequence
  Synthetic Polypeptide
  MISC_FEATURE (16)..(16): d-Ala
  MISC_FEATURE (18)..(18): PEG12
RVIRACLGVGLLGDLAGK

SEQ ID NO: 31 - protein, 19 aa, Artificial Sequence
  Synthetic Polypeptide
  MISC_FEATURE (1)..(1): Xaa is absent, K or M
  MISC_FEATURE (3)..(3): Xaa is V or d(A)
  MISC_FEATURE (6)..(6): Xaa is M, A or Nle
  MISC_FEATURE (7)..(7): Xaa is C or S
  MISC_FEATURE (9)..(9): Xaa is G or N
  MISC_FEATURE (10)..(10): Xaa is V or N
  MISC_FEATURE (12)..(12): Xaa is N or L
  MISC_FEATURE (13)..(13): Xaa is L, N or E
  MISC_FEATURE (15)..(15): Xaa is D or E
  MISC_FEATURE (16)..(16): Xaa is absent or L
  MISC_FEATURE (17)..(17): Xaa is absent or A or dA
  MISC_FEATURE (17)..(17): Xaa is absent when X16 is absent
  MISC_FEATURE (18)..(18): Xaa is absent when X16 is absent
  MISC_FEATURE (18)..(18): Xaa is G when X17 is A
  MISC_FEATURE (18)..(18): Xaa is absent or G or E or absent when X18 is dA
  MISC_FEATURE (19)..(19): Xaa is absent when X16 is absent or when X17 is absent or when X18 is absent or when X18 is E
  MISC_FEATURE (19)..(19): Xaa is absent or K when X18 is G
XRXIRXXLXXGXXGXXXXX

SEQ ID NO: 32 - protein, 18 aa, Artificial Sequence
  Synthetic Polypeptide
  MISC_FEATURE (5)..(5): Nle
  DISULFID (6)..(6)
  MISC_FEATURE (16)..(16): D-Ala
  MISC_FEATURE (18)..(18): PEG12
RVIRLCLGVGLLGDLAGK

SEQ ID NO: 33 - protein, 18 aa, Artificial Sequence
  Synthetic Polypeptide
  MISC_FEATURE (5)..(5): Nle
  MISC_FEATURE (16)..(16): D-Ala
RVIRKCLGVGLLGDLAGK

SEQ ID NO: 34 - protein, 18 aa, Artificial Sequence
  Synthetic Polypeptide
  MISC_FEATURE (5)..(5): Nle
  MISC_FEATURE (16)..(16): D-Ala
  MISC_FEATURE (18)..(18): PEG12
RVIRLCLGVGLLGDLAGK

SEQ ID NO: 35 - protein, 18 aa, Artificial Sequence
  Synthetic Polypeptide
  MISC_FEATURE (16)..(16): D-Ala
RVIRACLGVGLLGDLAGK

SEQ ID NO: 36 - protein, 18 aa, Artificial Sequence
  Synthetic Polypeptide
  DISULFID (6)..(6)
  MISC_FEATURE (36)..(36): PEG12
RVIRACLGVGLLGDLAGK

SEQ ID NO: 37 - protein, 18 aa, Artificial Sequence
  Synthetic Polypeptide
RVIRACLGVGLLGDLAGK

SEQ ID NO: 38 - protein, 18 aa, Artificial Sequence
  Synthetic Polypeptide
  MISC_FEATURE (18)..(18): PEG12
RVIRACLGVGLLGDLAGK

SEQ ID NO: 39 - protein, 11 aa, Artificial Sequence
  Synthetic Polypeptide
YARAAARQARA

SEQ ID NO: 40 - protein, 7 aa, Artificial Sequence
  Synthetic Polypeptide
YGRKKRR

SEQ ID NO: 41 - protein, 11 aa, Artificial Sequence
  Synthetic Polypeptide
YGRKKRRQRRR

SEQ ID NO: 42 - protein, 5 aa, Artificial Sequence
  Synthetic Polypeptide
RRQRR